(19) United States
(12) Patent Application Publication
(10) Pub. No.: US 2024/0221904 A1
(43) Pub. Date: Jul. 4, 2024

(54) INFORMATION PROCESSING DEVICE AND NON-TRANSITORY COMPUTER-READABLE STORAGE MEDIUM STORING PROGRAM

(71) Applicant: SEIKO EPSON CORPORATION, Tokyo (JP)

(72) Inventors: Shinya SATO, Matsumoto-shi (JP); Yuya OZAWA, Azumino-shi (JP)

(73) Assignee: SEIKO EPSON CORPORATION, Tokyo (JP)

(21) Appl. No.: 18/397,326

(22) Filed: Dec. 27, 2023

(30) Foreign Application Priority Data
Dec. 28, 2022 (JP) 2022-211857

(51) Int. Cl. G16H 20/60; G16H 15/00; G16H 50/20

(52) U.S. Cl. CPC G16H 20/60; G16H 15/00; G16H 50/20

(57) ABSTRACT

An information processing device of one aspect of the present disclosure includes an exercise information acquiring unit configured to acquire first exercise information related to an exercise performed by a subject person, a diagnosis information acquiring unit configured to acquire first diagnosis information of the subject person, a goal setting unit configured to set a first weight loss goal based on the first diagnosis information, a storage unit configured to store in advance a first trained model, the first trained model being configured to output, as a content of first guidance, a first advice candidate expected to be valid to achieve the first weight loss goal among a plurality of first advice candidates when the first exercise information and the first weight loss goal are input, and a report generation unit configured to generate a first guidance report including the content of the first guidance.

The present application is based on, and claims priority from JP Application Serial Number 2022-211857, filed Dec. 28, 2022, the disclosure of which is hereby incorporated by reference herein in its entirety.

### BACKGROUND

### 1. Technical Field

The present disclosure relates to an information processing device and a non-transitory computer-readable storage medium storing a program.

### 2. Related Art

JP-A-2021-12617 discloses a health support system that provides a diagram visualizing exercise information acquired by a measurement terminal worn on a user, and a message given by a supporter based on the exercise information.

In the health support system disclosed in JP-A-2021-12617, the contents of the messages provided to the user differ depending on the view of each supporter, and consequently effective messages may not necessarily be provided to the user even when the user performs the same exercise.

### SUMMARY

An information processing device of one aspect of the present disclosure includes an exercise information acquiring unit configured to acquire first exercise information related to an exercise performed by a subject person who receives health guidance, a diagnosis information acquiring unit configured to acquire first diagnosis information including a result of a first health checkup of the subject person, a goal setting unit configured to set a first weight loss goal of the subject person based on the first diagnosis information, a storage unit configured to store in advance a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input, and a report generation unit configured to input the first exercise information and the first weight loss goal to the first trained model, and generate a first guidance report including the content of the first guidance output from the first trained model.

A program of one aspect of the present disclosure is configured to cause a computer to execute acquiring first exercise information related to an exercise performed by a subject person who receives a health guidance, acquiring first diagnosis information including a result of a first health checkup of the subject person, setting a first weight loss goal of the subject person based on the first diagnosis information, reading from a storage unit a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input, and inputting the first exercise information and the first weight loss goal to the first trained model, and generating a first guidance report including the content of the first guidance output from the first trained model.

### BRIEF DESCRIPTION OF THE DRAWING

FIG. 1 is a block diagram illustrating a schematic configuration of an information processing device of a first embodiment.

FIG. 2 is a diagram illustrating an example of measurement data of a heart rate of a subject person.

FIG. 3 is a diagram illustrating an example of exercise intensity criteria.

FIG. 4 is a diagram illustrating an example of exercise frequency criteria.

FIG. 5 is a diagram illustrating an example of exercise time period criteria.

FIG. 6 is a diagram illustrating a first example of an exercise pattern.

FIG. 7 is a diagram illustrating a second example of an exercise pattern.

FIG. 8 is a diagram illustrating a third example of an exercise pattern.

FIG. 9 is a diagram illustrating a fourth example of an exercise pattern.

FIG. 10 is a diagram illustrating an example of a first weight loss goal set for a subject person.

FIG. 11 is a diagram illustrating an example of a first advice candidate.

FIG. 12 is a first diagram illustrating a procedure of generating a first trained model.

FIG. 13 is a second diagram illustrating a procedure of generating the first trained model.

FIG. 14 is a flowchart illustrating a process of generating a guidance report of the first embodiment.

FIG. 15 is a block diagram illustrating a schematic configuration of an information processing device of a second embodiment.

FIG. 16 is a diagram illustrating an example of a second advice candidate.

FIG. 17 is a first diagram illustrating a procedure of generating a second trained model.

FIG. 18 is a second diagram illustrating a procedure of generating the second trained model.

FIG. 19 is a flowchart illustrating a process of generating a guidance report of the second embodiment.

FIG. 20 is a block diagram illustrating a schematic configuration of an information processing device of a third embodiment.

FIG. 21 is a diagram illustrating an example of a third advice candidate.

FIG. 22 is a first diagram illustrating a procedure of generating a third trained model.

FIG. 23 is a second diagram illustrating a procedure of generating the third trained model.

FIG. 24 is a flowchart illustrating a process of generating a guidance report of the third embodiment.

### DESCRIPTION OF EMBODIMENTS

Embodiments of the present disclosure are described below with reference to the accompanying drawings. Note that in each drawing described below, each member may not be drawn to scale for illustration of each member in recognizable sizes.

### First Embodiment

First, a first embodiment is described.

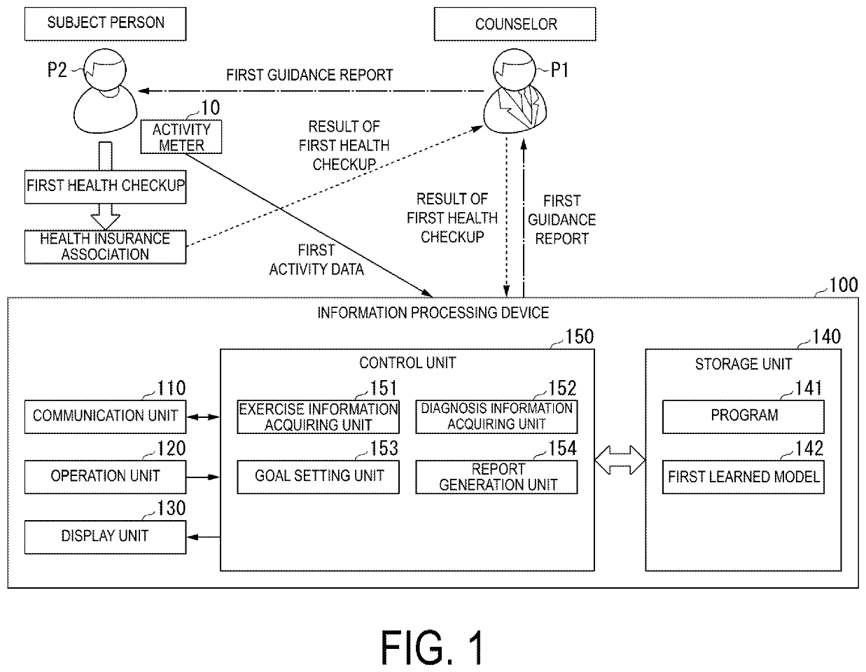

FIG. 1 is a block diagram illustrating a schematic configuration of an information processing device 100 of the first embodiment. The information processing device 100 is an apparatus for supporting health guidance performed by a counselor P1 for a subject person P2. For example, the information processing device 100 is a PC (Personal Computer). The information processing device 100 acquires first activity data of the subject person P2 from an activity meter 10 worn on the subject person P2 who takes the health guidance.

The activity meter 10, worn on the subject person P2, measures the activity amount of the subject person P2. For example, the activity meter 10 measures at least one of number of steps, distance exercised, calories burned, and heart rate as the activity amount of the subject person P2. The activity meter 10 transmits data including the measurement data of the activity amount and the identification information of the subject person P2 to the information processing device 100 as the first activity data of the subject person P2. For example, the identification information of the subject person P2 includes the insurer number of the health insurance association in which the subject person P2 is a member, and the insured person number of the subject person P2.

The subject person P2 operates the activity meter 10 after going about his or her daily life while wearing the activity meter 10 for the measurement period specified by the counselor P1 before the first guidance report is provided, and thus transmits first activity data including the measurement data of the activity amount obtained in the measurement period to the information processing device 100. That is, the first activity data transmitted from the activity meter 10 to the information processing device 100 includes the measurement data of the activity amount obtained in the measurement period before the first guidance report is provided to the subject person P2. For example, a first health checkup is a health checkup that the subject person P2 takes before his or her first meeting with the counselor P1.

Note that when the activity meter 10 cannot directly transmit the first activity data to the information processing device 100, the activity meter 10 may transmit the first activity data to the information processing device 100 via a user terminal such as a smartphone and a tablet terminal that can be used by the subject person P2. The above-described activity meter 10 is an example of the measurement device.

When the subject person P2 takes the first health checkup, the result of the first health checkup of the subject person P2 is provided from the health insurance association to the counselor P1. The method for providing the result of the first health checkup to the counselor P1 is not limited. For example, the subject person P2 himself/herself may provide a printed material of the result of the first health checkup to the counselor P1. Alternatively, the subject person P2 may transmit an e-mail with an electronic file of the result of the first health checkup attached to it, to a smartphone, a tablet terminal or the like that can be used by the counselor P1. In either way, the result of the first health checkup in linkage with the identification information of the subject person P2 is provided to the counselor P1.

The counselor P1 inputs the identification information of the subject person P2 and the result of the first health checkup to the information processing device 100 by operating the information processing device 100. The information processing device 100 generates the first guidance report including the content of the first guidance for the subject person P2 on the basis of the first activity data of the subject person P2 received from the activity meter 10 and the result of the first health checkup of the subject person P2 input by the counselor P1.

The information processing device 100 provides the generated first guidance report to the counselor P1. The method for providing the first guidance report to the counselor P1 is not limited. For example, in the case where a printer is coupled to the information processing device 100, the information processing device 100 may provide a printed material of the content of the first guidance report to the counselor P1 through the printer. Alternatively, the information processing device 100 may transmit an e-mail with an electronic file of the first guidance report attached to it, to a smartphone, a tablet terminal or the like that can be used by the counselor P1.

After confirming the content of the first guidance report, the counselor P1 provides the first guidance report to the subject person P2. The method for providing the first guidance report to the subject person P2 is not limited. For example, in the case where the counselor P1 has obtained the first guidance report as a printed material, a printed material of the first guidance report may be provided to the subject person P2. Alternatively, in the case where the counselor P1 has obtained the first guidance report as an electronic file, an e-mail with an electronic file of the first guidance report attached to it may be transmitted to the user terminal of the subject person P2.

A configuration of the information processing device 100 is elaborated below. As illustrated in FIG. 1, the information processing device 100 includes a communication unit 110, an operation unit 120, a display unit 130, a storage unit 140, and a control unit 150.

The communication unit 110 is a communication interface apparatus that communicates with an external apparatus. For example, the communication unit 110 communicates with the activity meter 10. In addition, the communication unit 110 may communicate with the activity meter 10 through the user terminal of the subject person P2 in the case where it cannot directly communicate with the activity meter 10. The communication unit 110 outputs, to the control unit 150, the first activity data of the subject person P2 received from the activity meter 10.

The operation unit 120 is an apparatus that receives inputting operation of the counselor P1 performed on the information processing device 100. For example, the operation unit 120 is an input apparatus such as a keyboard and a mouse. The operation unit 120 outputs an electric signal generated by an operation of a keyboard, a mouse and the like to the control unit 150 as an operation signal. When receiving an inputting operation of the identification information of the subject person P2 and the result of the first health checkup from the counselor P1, the operation unit 120 outputs an electric signal generated by the inputting operation to the control unit 150 as an operation signal.

The display unit 130 displays a predetermined image under the control of the control unit 150. The display unit 130 is, for example, a liquid crystal monitor, an organic EL (Electro Luminescence) monitor or the like.

The storage unit 140 stores programs, data and the like required for the control unit 150 to execute various processes. For example, the storage unit 140 includes nonvolatile storage apparatuses such as a HDD (Hard Disk Drive), a flash memory, and a ROM (Read Only Memory), and volatile storage apparatuses such as a RAM (random access memory).

The storage unit 140 stores a program 141, a first trained model 142 and the like. The program 141 includes programs for the control unit 150 to execute a guidance report generation process described later, and the like, in addition to the OS (Operating System) and application programs. In the following description, the program for the control unit 150 to execute the guidance report generation process is referred to as guidance report generation program. Details of the first trained model 142 are described later.

In accordance with the program 141 stored in the storage unit 140, the control unit 150 determines the entire operation of the information processing device 100. For example, the control unit 150 is a processor such as a CPU (Central Processing Unit). The control unit 150 includes an exercise information acquiring unit 151, a diagnosis information acquiring unit 152, a goal setting unit 153, and a report generation unit 154. These components are functions that are achieved when the control unit 150 executes the above-mentioned guidance report generation program.

Some or all of the functions of the control unit 150 may be achieved by hardware such as a LSI (Large Scale Integration), an ASIC (Application Specific Integrated Circuit), a FPGA (Field-Programmable Gate Array), and a GPU (Graphics Processing Unit), or a combination of software and hardware.

On the basis of the first activity data received from the activity meter 10 through the communication unit 110, the exercise information acquiring unit 151 acquires first exercise information related to the exercise performed by the subject person P2 who takes the health guidance before the first guidance report is provided. For example, on the basis of the measurement data of the activity amount, i.e., the measurement data of the activity amount obtained in the measurement period before the first guidance report is provided to the subject person P2, the exercise information acquiring unit 151 acquires the exercise pattern of the subject person P2 as the first exercise information.

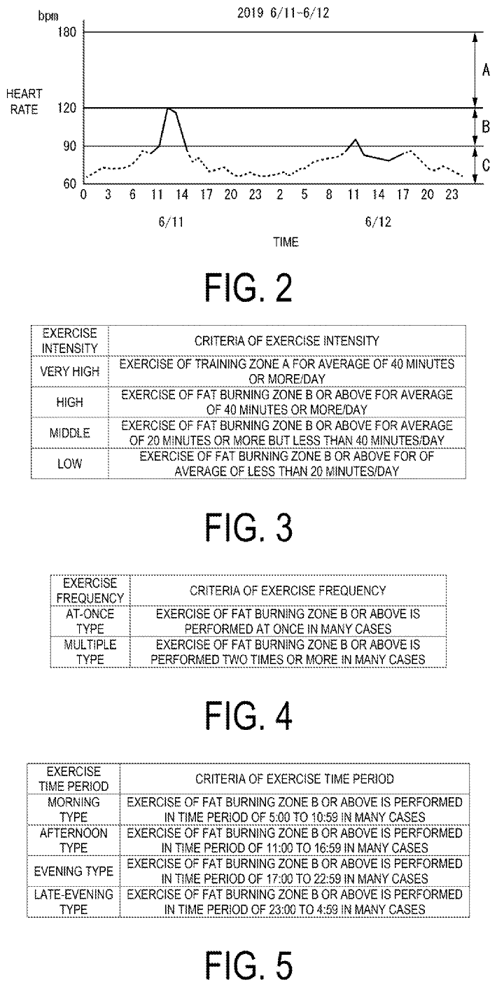

More specifically, on the basis of the measurement data of the activity amount included in the first activity data, the exercise information acquiring unit 151 acquires at least one of the exercise intensity, the exercise frequency, and the exercise time period, as data representing the exercise pattern. An example of a method for acquiring the exercise intensity, the exercise frequency, and the exercise time period by the exercise information acquiring unit 151 is described below. Note that in the following description, terms training zone A, fat burning zone B, and warm-up zone C are frequently used, and therefore definitions of these terms are described first with reference to FIG. 2.

The measurement data of the activity amount includes at least heart rate measurement data. FIG. 2 is a diagram illustrating an example of heart rate measurement data of the subject person P2. In FIG. 2, the ordinate indicates the heart rate, and the abscissa indicates the time. For example, FIG. 2 illustrates heart rate measurement data of the subject person P2 obtained in a measurement period from Jun. 11, 2019 to Jun. 12, 2019.

As illustrated in FIG. 2, in this embodiment, a region where the heart rate is 120 bpm or greater is defined as the training zone A that contributes to both fat burning and physical fitness. For example, when the subject person P2 is exercising to the extent that the subject person P2 is out of breath, such as running, the heart rate of the subject person P2 is likely to reach the training zone A.

In addition, in this embodiment, the region where the heart rate is 90 bpm or greater but is less than 120 bpm is defined as the fat burning zone B that contributes to fat burning. For example, when the subject person P2 is exercising to the extent that the subject person P2 is a little out of breath such as walking quickly or going up and down stairs, the heart rate of the subject person P2 is likely to reach the fat burning zone B.

Further, in this embodiment, the region where the heart rate is less than 90 bpm is defined as the warm-up zone C that less contributes to fat burning. For example, when the subject person P2 is lightly exercising to the extent that the subject person P2 is not out of breath, such as walking at less than normal walking speed, the heart rate of the subject person P2 is likely to remain in the warm-up zone C.

The terms training zone A, fat burning zone B, and warm-up zone C are defined as described above.

The exercise information acquiring unit 151 determines at least one of the exercise intensity, the exercise frequency, and the exercise time period on the basis of the heart rate measurement data and predetermined criteria. FIG. 3 is a diagram illustrating an example of exercise intensity criteria. The exercise information acquiring unit 151 determines which of “very high”, “high”, “medium”, and “low” corresponds to the exercise intensity of the subject person P2 on the basis of the heart rate measurement data and the exercise intensity criteria.

For example, as illustrated in FIG. 3, in the case where the subject person P2 performs an exercise of the training zone A for an average of 40 minutes or more per day, the exercise information acquiring unit 151 determines that the exercise intensity of the subject person P2 is “very high”. In addition, in the case where the subject person P2 performs an exercise of the fat burning zone B or above for an average of 40 minutes or more per day, the exercise information acquiring unit 151 determines that the exercise intensity of the subject person P2 is “high”.

In addition, in the case where the subject person P2 performs an exercise of the fat burning zone B or above for an average of at least 20 minutes but less than 40 minutes per day, the exercise information acquiring unit 151 determines that the exercise intensity of the subject person P2 is “medium”. Further, in the case where the subject person P2 performs an exercise of the fat burning zone B or above for an average of less than 20 minutes per day, the exercise information acquiring unit 151 determines that the exercise intensity of the subject person P2 is “low”. In the example illustrated in FIG. 2, the exercise intensity on June 11 is determined to be “high”, and the exercise intensity on June 12 is determined to be “low”.

FIG. 4 is a diagram illustrating an example of exercise frequency criteria. The exercise information acquiring unit 151 determines which of “at-once type” and “multiple type” corresponds to the exercise frequency of the subject person P2 on the basis of the heart rate measurement data and the exercise frequency criteria.

For example, as illustrated in FIG. 4, in the case where the subject person P2 performs an exercise of the fat burning zone B or above per day at once in many cases, the exercise information acquiring unit 151 determines that the exercise frequency of the subject person P2 is “at-once type”. In addition, in the case where the subject person P2 performs an exercise of the fat burning zone B or above two times or more per day in many cases, the exercise information acquiring unit 151 determines that the exercise frequency of the subject person P2 is “multiple type”. In the example illustrated in FIG. 2, the exercise frequency on June 11 and the exercise frequency on June 12 are both determined to be “at-once type”.

FIG. 5 is a diagram illustrating an example of the exercise time period criteria. The exercise information acquiring unit 151 determines which of “morning type”, “afternoon type”, “evening type”, and “late-evening type” corresponds to the exercise time period of the subject person P2 on the basis of the heart rate measurement data and the exercise time period criteria.

For example, as illustrated in FIG. 5, in the case where the subject person P2 performs an exercise of the fat burning zone B or above in the time period from 5:00 to 10:59 in many cases, the exercise information acquiring unit 151 determines that the exercise time period of the subject person P2 is “morning type”. In addition, in the case where the subject person P2 performs an exercise of the fat burning zone B or above in a time period from 11:00 to 16:59 in many cases, the exercise information acquiring unit 151 determines that the exercise time period of the subject person P2 is “afternoon type”.

In addition, in the case where the subject person P2 performs an exercise of the fat burning zone B or above in a time period from 17:00 to 22:59 in many cases, the exercise information acquiring unit 151 determines that the exercise time period of the subject person P2 is “evening type”. Further, in the case where the subject person P2 performs an exercise of the fat burning zone B or above in a time period from 23:00 to 4:59 in many cases, the exercise information acquiring unit 151 determines that the exercise time period of the subject person P2 is “late-evening type”.

In the above-described manner, the exercise information acquiring unit 151 acquires at least one of the exercise intensity, the exercise frequency, and the exercise time period as data representing the exercise pattern on the basis of the measurement data of the activity amount.

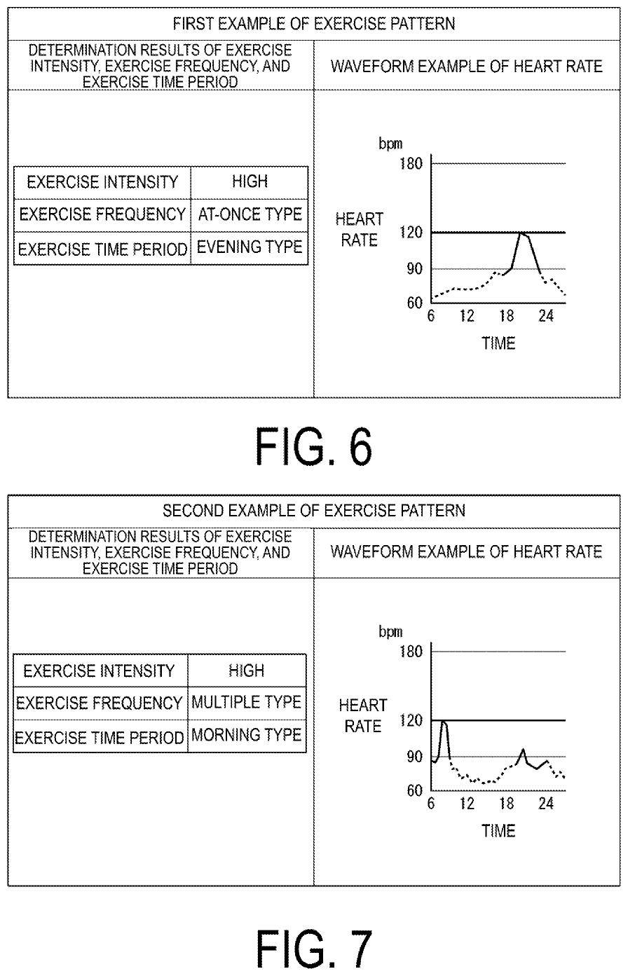

FIG. 6 is a diagram illustrating a first example of an exercise pattern. FIG. 6 illustrates determination results of the exercise intensity, the exercise frequency, and the exercise time period in a first example of the exercise pattern, and a waveform example of the heart rate for reference purposes. In the first example of the exercise pattern, the exercise intensity is “high”, the exercise frequency is “at-once type”, and the exercise time period is “evening type”. The first example of the exercise pattern is a pattern in which the subject person P2 performs activities such that the exercise intensity is high in the evening time period. The subject person P2 corresponding to the first example of the exercise pattern is, for example, a person who goes for a run or exercises at the gym after work.

FIG. 7 is a diagram illustrating a second example of an exercise pattern. FIG. 7 illustrates determination results of the exercise intensity, the exercise frequency, and the exercise time period in the second example of the exercise pattern, and a waveform example of the heart rate for reference purposes. In the second example of the exercise pattern, the exercise intensity is “high”, the exercise frequency is “multiple type”, and the exercise time period is “morning type”. The second example of the exercise pattern is a pattern in which the subject person P2 performs activities such that the exercise intensity is high in the morning and evening time periods. The subject person P2 corresponding to the second example of the exercise pattern is, for example, a person who walks in the morning, and performs light exercise in the evening.

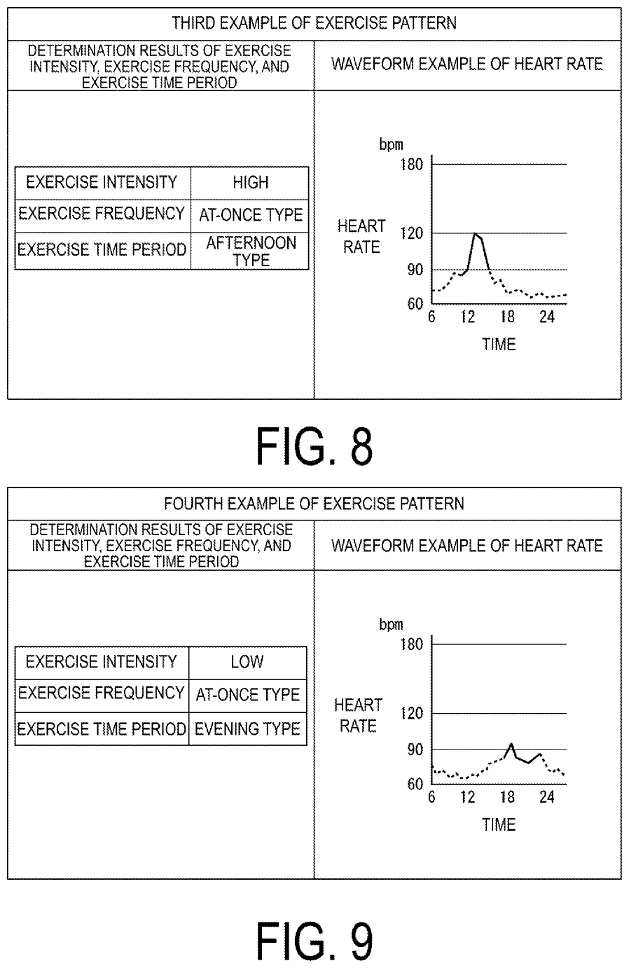

FIG. 8 is a diagram illustrating a third example of an exercise pattern. FIG. 8 illustrates determination results of the exercise intensity, the exercise frequency, and the exercise time period in the third example of the exercise pattern, and a waveform example of the heart rate for reference purposes. In the third example of the exercise pattern, the exercise intensity is “high”, the exercise frequency is “at-once type”, and the exercise time period is “afternoon type”. The third example of the exercise pattern is a pattern in which the subject person P2 performs activities such that the exercise intensity is high in a daytime period. The subject person P2 corresponding to the third example of the exercise pattern is, for example, a person who walks during lunch breaks or the like.

FIG. 9 is a diagram illustrating a fourth example of an exercise pattern. FIG. 9 illustrates determination results of the exercise intensity, the exercise frequency, and the exercise time period in the fourth example of the exercise pattern, and a waveform example of the heart rate for reference purposes. In the fourth example of the exercise pattern, the exercise intensity is “low”, the exercise frequency is “at-once type”, and the exercise time period is “evening type”. The fourth example of the exercise pattern is a pattern in which the subject person P2 performs activities such that the exercise intensity is not high in the evening time period. The subject person P2 corresponding to the fourth example of the exercise pattern is, for example, a person who occasionally exercises after work.

Note that the exercise information acquiring unit 151 may not acquire all of the exercise intensity, the exercise frequency, and the exercise time period as data representing the exercise pattern of the subject person P2. The exercise information acquiring unit 151 may acquire at least one of the exercise intensity, the exercise frequency, and the exercise time period as data representing the exercise pattern of the subject person P2.

The exercise information acquiring unit 151 is described above. In the following description, the diagnosis information acquiring unit 152 is described with reference to FIG. 1 again. As already described above, when receiving from the counselor P1 an inputting operation of the identification information of the subject person P2 and the result of the first health checkup, the operation unit 120 outputs the electric signal generated by the inputting operation to the control unit 150 as an operation signal. The diagnosis information acquiring unit 152 acquires first diagnosis information including the result of the first health checkup of the subject person P2 on the basis of the operation signal output from the operation unit 120.

For example, the first diagnosis information includes, as a result of the first health checkup, diagnosis results such as the weight, chest circumference, waist circumference, neutral fat, fasting blood glucose level, blood pressure (systolic blood pressure and diastolic blood pressure), and BMI (Body Mass Index) of the subject person P2. These diagnosis items are merely examples, and the result of the first health checkup may include diagnosis results of other diagnosis items.

The goal setting unit 153 sets the first weight loss goal of the subject person P2 on the basis of the first diagnosis information. For example, the goal setting unit 153 sets the first weight loss goal such that the diagnosis results of the waist circumference, neutral fat, fasting blood glucose level, and blood pressure of the subject person P2 are smaller than the diagnosis reference values for metabolic syndrome. As an example, the diagnosis reference values for metabolic syndrome related to the waist circumference, neutral fat, fasting blood glucose level, and blood pressure are as follows.

### Diagnosis Reference Values for Metabolic Syndrome

(a) Waist circumference: 85 cm or more for men, 90 cm or greater for women
(b) Neutral fat: 150 mg/dl or greater
(c) Fasting blood glucose level: 110 mg/dl or greater
(d) Blood pressure: 130 mm Hg or greater for systolic blood pressure, and 85 mm Hg or greater for diastolic blood pressure

When the diagnosis result of the waist circumference and the diagnosis results of at least two of the neutral fat, fasting blood glucose level, and blood pressure correspond to the diagnosis reference values for metabolic syndrome, the subject person P2 is diagnosed with metabolic syndrome, and subjected to specific health guidance.

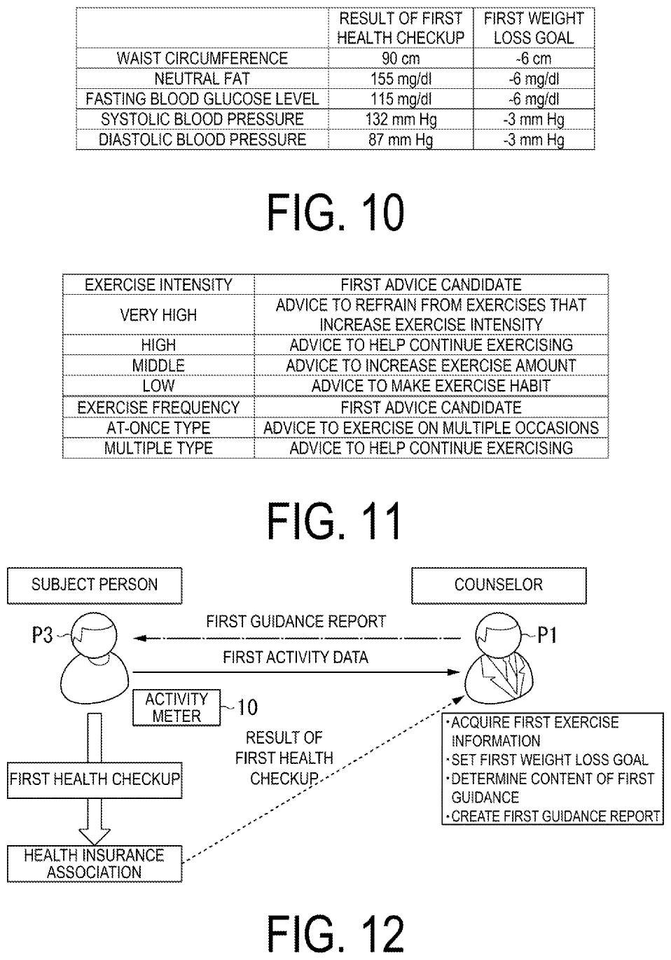

FIG. 10 is a diagram illustrating an example of a first weight loss goal set for the subject person P2. In the example illustrated in FIG. 10, the subject person P2 is a 40 year old male with a diagnosis result of the waist circumference of 90 cm, a diagnosis result of the neutral fat of 155 mg/dl, a diagnosis result of the fasting blood glucose level of 115 mg/dl, a diagnosis result of the systolic blood pressure of 132 mm Hg, and a diagnosis result of the diastolic blood pressure of 87 mm Hg.

In this case, the goal setting unit 153 sets the first weight loss goal of the waist circumference to −6 cm, the first weight loss goal of the neutral fat to −6 mg/dl, the first weight loss goal of the fasting blood glucose level to −6 mg/dl, the first weight loss goal of the systolic blood pressure to −3 mm Hg, and the first weight loss goal of the diastolic blood pressure to −3 mm Hg.

In addition, the first weight loss goal of the subject person P2 set by the goal setting unit 153 may be a goal related to the weight and the girth of the abdomen of the subject person P2. The goal related to the weight of the subject person P2, for example, may be a weight loss for each month of about several % from the weight of the subject person P2 at the time of the first health checkup, or may be a weight with which the BMI of the subject person P2 is 25 or smaller. By setting the first weight loss goal to the goal related to the weight of the subject person P2, the subject person P2 can easily confirm the results of the activities performed by the subject person P2, and can easily maintain the motivation for the activities.

Referring to FIG. 1 again, the report generation unit 154 inputs the first exercise information and the first weight loss goal to the first trained model 142, and generates the first guidance report including the content of the first guidance output from the first trained model 142. The first trained model 142 is a mathematical model that outputs the first advice candidate expected to be valid to achieve the first weight loss goal among a plurality of first advice candidates based on the first exercise information as the content of the first guidance for the subject person P2 when the first exercise information and the first weight loss goal are input.

For example, the first advice candidate includes an advice related to an exercise. For example, an advice related to an exercise is an introduction to the exercise performed by the subject person P2, an advice on the exercise performed by the subject person P2, and an advice on how to walk in daily life and the like.

FIG. 11 is a diagram illustrating an example of the first advice candidate. As illustrated in FIG. 11, for example, the first advice candidate corresponding to the exercise intensity of “very high” includes an advice for the subject person P2 to refrain from exercises that increase the exercise intensity. The reason for this is that the subject person P2 subjected to the health guidance has not exercised much in the daily life before the health guidance period, and therefore the risk of injury is high if the subject person P2 suddenly performs very high-intensity exercise.

For example, the first advice candidate corresponding to the exercise intensity of “high” includes an advice to help the subject person P2 continue exercising. The reason for this is that the subject person P2 whose exercise intensity is “high” is able to perform exercises that ensure the targeted exercise amount in the daily life.

For example, the first advice candidate corresponding to the exercise intensity of “medium” includes an advice to increase the exercise amount of the subject person P2. The reason for this is that the subject person P2 whose exercise intensity is “medium” is not able to perform exercises that ensure the targeted exercise amount in the daily life.

For example, the first advice candidate corresponding to the exercise intensity of “low” includes an advice for the subject person P2 to make exercise a habit. The reason for this is that the subject person P2 whose exercise intensity is “low” may not be able to continue and follow the advice for a long time even if the subject person P2 receives the advice to increase the exercise intensity, and needs to make exercise a habit first.

For example, the first advice candidate corresponding to the exercise frequency of “at-once type” includes an advice for the subject person P2 to perform the exercise on multiple occasions. The reason for this is that the subject person P2 whose exercise frequency is “at-once type” is unbalanced between the exercise amount on days when exercise is performed and the exercise amount on days when exercise is not performed in the daily life.

For example, the first advice candidate corresponding to the exercise frequency of “multiple type” includes an advice to help the subject person P2 continue exercising. The reason for this is that the subject person P2 whose exercise frequency is “multiple type” can easily maintain the exercise amount because, even when the exercise is not performed in a certain time period, the exercise is performed in other time periods in the daily life.

Note that the first advice candidate may include not only the above-described advices related to the exercise but also an advice related to diet and sleep. For example, the advice related to diet is an advice related to the amount of food, the time of day to eat, and the nutritional balance of the meal. For example, the advice related to sleep is an advice on waking and sleeping time. In addition, the first guidance report may include other information such as the result of the first health checkup and the first weight loss goal in addition to the content of the first guidance output from the first trained model 142.

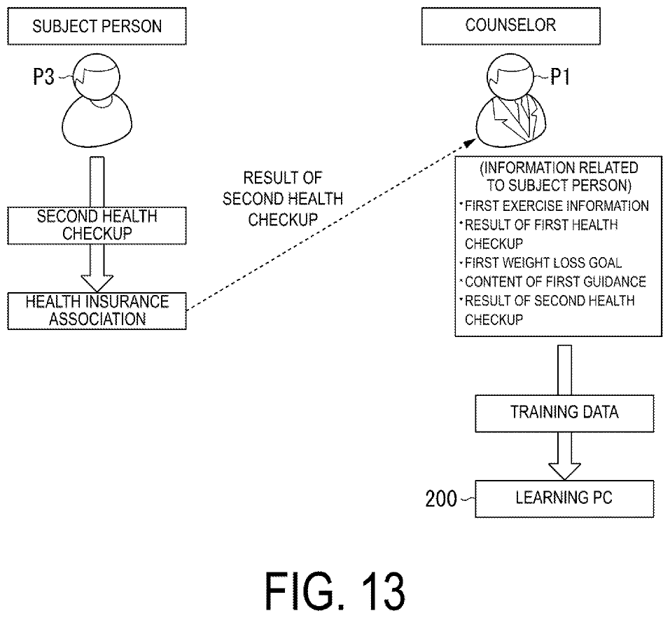

The first trained model 142 is a mathematical model generated through machine learning such as deep learning, for example. A procedure of generating the first trained model 142 is described below with reference to FIGS. 12 and 13.

As illustrated in FIG. 12, a subject person P3 different from the subject person P2 provides the first activity data obtained by the activity meter 10 to the counselor P1 after going about his or her daily life while wearing the activity meter 10 for the measurement period specified by the counselor P1 before the first guidance report is provided. Further, the result of the first health checkup taken by the subject person P3 in advance is provided from the health insurance association to the counselor P1.

The counselor P1 acquires the first exercise information of the subject person P3 on the basis of the first activity data received from the subject person P3. More specifically, the counselor P1 acquires the exercise pattern of the subject person P2 as the first exercise information on the basis of the measurement data of the activity amount included in the first activity data received from the subject person P3.

In addition, the counselor P1 sets the first weight loss goal of the subject person P3 on the basis of the result of the first health checkup provided from the health insurance association. To set a goal related to the diagnosis reference for metabolic syndrome, the counselor P1 sets the first weight loss goal such that the diagnosis results of the waist circumference, neutral fat, fasting blood glucose level, and blood pressure of the subject person P3 are smaller than the diagnosis reference values for metabolic syndrome.

Then, on the basis of the first exercise information of the subject person P3, the counselor P1 determines the first advice candidate for achieving the first weight loss goal among a plurality of the first advice candidates as those illustrated in FIG. 11 as the content of the first guidance for the subject person P3. Here, the counselor P1 may not necessarily determine the content of the first guidance completely matching the correspondence relationship of the first exercise information and the first advice candidate as that illustrated in FIG. 11.

For example, even in the case where the exercise intensity of the subject person P3 is “high”, the counselor P1 may determine the first advice candidate corresponding to the exercise intensity of “very high” as the content of the first guidance in consideration of the first weight loss goal and the like. Alternatively, in the case where the exercise intensity of the subject person P3 is “high”, the counselor P1 may determine the first advice candidate corresponding to the exercise intensity of “high” as the content of the first guidance in accordance with the relationship illustrated in FIG. 11 in consideration of the first weight loss goal and the like. In this manner, the correspondence relationship of the exercise pattern and the first advice candidate illustrated in FIG. 11 is used for reference only, and the first guidance of different contents may be determined for the same exercise pattern in accordance with the first weight loss goal of the subject person P3.

The counselor P1 creates the first guidance report including the content of the first guidance determined in the above-described manner, the result of the first health checkup of the subject person P3, and the first weight loss goal. The counselor P1 provides the created first guidance report to the subject person P3.

As illustrated in FIG. 13, the subject person P3 takes the second health checkup after going about his or her daily life in accordance with the first guidance report received from the counselor P1. For example, the second health checkup is a health checkup taken by the subject person P3 after receiving the first guidance report. The second health checkup may be the same diagnosis as the first health checkup, or may be a diagnosis simpler than the first health checkup such as a diagnosis of measuring the weight and the girth of the abdomen. In the case where the second health checkup is a diagnosis of measuring the weight and the girth of the abdomen, the goal set as the first weight loss goal is preferably a goal related to the weight and the girth of the abdomen.

When the subject person P3 takes the second health checkup, the result of the second health checkup of the subject person P3 is provided to the counselor P1. Here, the counselor P1 may receive the result of the second health checkup of the subject person P3 via the health insurance association, or not via the health insurance association.

The counselor P1 confirms whether the first weight loss goal of the subject person P3 has been achieved by the content of the first guidance in the first guidance report on the basis of the result of the second health checkup of the subject person P3. Specifically, in the case where the first weight loss goal is set as a goal related to the diagnosis reference for metabolic syndrome, the counselor P1 confirms whether the diagnosis results of the waist circumference, neutral fat, fasting blood glucose level, and blood pressure of the subject person P3 are smaller than the diagnosis reference values for metabolic syndrome among the results of the second health checkup.

In this manner, the counselor P1 acquires, as information related to the subject person P3, the first exercise information, the result of the first health checkup, the first weight loss goal, the content of the first guidance, and the result of the second health checkup. The result of the second health checkup is information representing whether the first weight loss goal of the subject person P3 has been achieved by the content of the first guidance in the first guidance report.

As is well known, supervised learning, as one of machine learning methods, is a method of learning the correlation of the learning data and the correct data by using a set of data to be input (learning data) and data to be output (correct data) as training data. Through supervised learning using large amount of such training data, a trained model is generated in which when unknown data is input, correct data corresponding to the input data is output.

The counselor P1 acquires the first exercise information and the first weight loss goal as data to be input, i.e., learning data from among information related to the subject person P3. In addition, the counselor P1 acquires the content of the first guidance that has succeeded in achieving the first weight loss goal of the subject person P3 as data to be output, i.e., correct data from among the information related to the subject person P3.

The counselor P1 acquires the above-described set of the learning data and the correct data as training data, and stores the acquired training data in linkage with the identification information of the subject person P3 in a learning PC 200. The learning PC 200 may be the same PC as the information processing device 100, or a PC different from the information processing device 100. Note that when the first weight loss goal of the subject person P3 has not been successfully achieved, the counselor P1 may not acquire the training data related to the subject person P3.

The same procedure as the above-mentioned procedure is sequentially applied to the subject person other than the subject person P3, and the training data linked with the identification information of the subject person other than the subject person P3 is sequentially stored in the learning PC 200. When the number of pieces of the training data stored in the learning PC 200 reaches a predetermined number, the counselor P1 operates the learning PC 200 such that supervised learning is performed on the basis of the stored training data.

As a result, a trained model is generated in which when a data including the first exercise information of an unknown subject person and the first weight loss goal of the unknown subject person is input, correct data corresponding to the input data, i.e., the first advice candidate expected to be valid to achieve the first weight loss goal of the unknown subject person among a plurality of the first advice candidates as those illustrated in FIG. 11 is output as the content of the first guidance for the unknown subject person.

The trained model generated by the learning PC 200 in this manner is the first trained model 142 of the first embodiment. Specifically, the first trained model 142 is a mathematical model in which when the first exercise information and the first weight loss goal of the subject person P2 as the unknown subject person are input, the first advice candidate expected to be valid to achieve the first weight loss goal of the subject person P2 among a plurality of the first advice candidates based on the first exercise information is output as the content of the first guidance for the subject person P2.

Next, an operation of the information processing device 100 having the above-mentioned configuration is described.

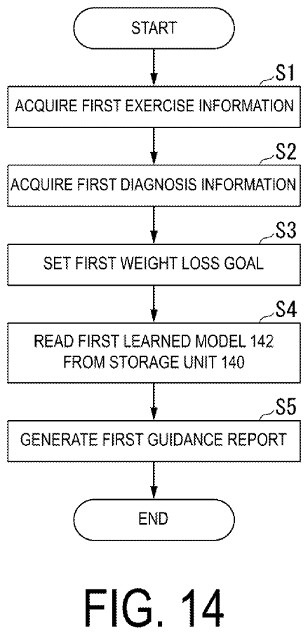

FIG. 14 is a flowchart illustrating a guidance report generation process executed by the control unit 150. When the control unit 150 detects a reception of an operation of instructing generation of the first guidance report from the counselor P1 on the basis of the operation signal output from the operation unit 120, the control unit 150 reads the guidance report generation program from the storage unit 140 and executes the program to execute the guidance report generation process illustrated in FIG. 14.

As illustrated in FIG. 14, when the guidance report generation process is started, first, the control unit 150 acquires the first exercise information related to the exercise performed by the subject person P2 who takes the health guidance before the first guidance report is provided on the basis of the first activity data received from the activity meter 10 through the communication unit 110 (step S1).

For example, at step S1, the control unit 150 acquires the exercise pattern of the subject person P2 as the first exercise information on the basis of the measurement data of the activity amount included in the first activity data, i.e., the measurement data of the activity amount obtained in the measurement period before the first guidance report is provided to the subject person P2. The process of this step S1 is the same process as the process executed by the exercise information acquiring unit 151, and therefore the description related to step S1 is omitted.

Subsequently, the control unit 150 acquires the first diagnosis information including the result of the first health checkup of the subject person P2 on the basis of the operation signal output from the operation unit 120 (step S2). For example, the first diagnosis information includes, as a result of the first health checkup, diagnosis results such as the weight, chest circumference, waist circumference, neutral fat, fasting blood glucose level, blood pressure (systolic blood pressure and diastolic blood pressure), and BMI of the subject person P2. The process of this step S2 is the same process as the process executed by the diagnosis information acquiring unit 152, and therefore the description related to step S2 is omitted.

Subsequently, the control unit 150 sets the first weight loss goal of the subject person P2 on the basis of the first diagnosis information (step S3). For example, at step S3, the control unit 150 sets the first weight loss goal such that the diagnosis results of the waist circumference, neutral fat, fasting blood glucose level, and blood pressure of the subject person P2 are smaller than the diagnosis reference values for metabolic syndrome. The process of this step S3 is the same process as the process executed by the goal setting unit 153, and therefore the description related to step S3 is omitted.

Subsequently, the control unit 150 reads the first trained model 142 from the storage unit 140 (step S4). Then, the control unit 150 inputs the first exercise information and the first weight loss goal to the first trained model 142, and generates the first guidance report including the content of the first guidance output from the first trained model 142 (step S5). The control unit 150 may generate the first guidance report including other information such as the result of the first health checkup and the first weight loss goal in addition to the content of the first guidance output from the first trained model 142. The process of this step S5 is the same process as the process executed by the report generation unit 154, and therefore the description related to step S5 is omitted.

### Effects of First Embodiment

As described above, the information processing device 100 of the first embodiment includes the exercise information acquiring unit 151 that acquires the first exercise information related to the exercise performed by the subject person P2 who takes the health guidance before the first guidance report is provided, the diagnosis information acquiring unit 152 that acquires the first diagnosis information including the result of the first health checkup of the subject person P2, the goal setting unit 153 that sets the first weight loss goal of the subject person P2 on the basis of the first diagnosis information, the storage unit 140 that stores in advance the first trained model 142 that outputs the first advice candidate expected to be valid to achieve the first weight loss goal among a plurality of the first advice candidates based on the first exercise information as the content of the first guidance for the subject person P2 when the first exercise information and the first weight loss goal are input, and the report generation unit 154 that inputs the first exercise information and the first weight loss goal to the first trained model 142, and generates the first guidance report including the content of the first guidance output from the first trained model 142.

As described above for the explanation of the generation procedure for the first trained model 142, when the counselor P1 creates the first guidance report, the content of the first guidance provided to the subject person may differ depending on the view of the counselor P1 even for the same exercise pattern. Such a content of the first guidance dependent on the view of the counselor P1 may or may not be effective for the subject person depending on the case.

On the other hand, the information processing device 100 of the first embodiment inputs the first exercise information and the first weight loss goal of the subject person P2 to the first trained model 142, and generates the first guidance report including the content of the first guidance output from the first trained model 142. The first trained model 142 is a mathematical model generated through machine learning in which when the first exercise information and the first weight loss goal of the subject person P2 are input, the first advice candidate expected to be valid to achieve the first weight loss goal among a plurality of the first advice candidates based on the first exercise information is output as the content of the first guidance for the subject person P2. Thus, regardless of the first exercise information and the first weight loss goal input to the first trained model 142, the correct content of the first guidance, i.e., the content of the first guidance that has succeeded in achieving the first weight loss goal in the past history is always output from the first trained model 142.

Therefore, according to the information processing device 100 of the first embodiment, the first guidance report including the content of the first guidance that is always effective for the first exercise information and the first weight loss goal of the subject person P2 can be provided to the subject person P2 regardless of the view of the counselor P1.

In the first embodiment, the first diagnosis information includes, as a result of the first health checkup, the diagnosis results of the waist circumference, neutral fat, fasting blood glucose level, and blood pressure of the subject person P2, and the goal setting unit 153 sets the first weight loss goal such that the diagnosis results of the waist circumference, neutral fat, fasting blood glucose level, and blood pressure are smaller than the diagnosis reference values for metabolic syndrome.

As described above, by setting the first weight loss goal such that the diagnosis results of the waist circumference, neutral fat, fasting blood glucose level, and blood pressure are smaller than the diagnosis reference values for metabolic syndrome, the first weight loss goal of the subject person P2 can be correctly set.

In the first embodiment, the exercise information acquiring unit 151 acquires the exercise pattern of the subject person P2 as the first exercise information on the basis of the measurement data of the activity amount obtained from the activity meter 10 worn on the subject person P2.

By acquiring the exercise pattern of the subject person P2 as the first exercise information on the basis of the measurement data of the activity amount in the above-described manner, the arithmetic processing load required for generating the first guidance report can be reduced in comparison with the case where the measurement data of the activity amount itself is acquired as the first exercise information.

In the first embodiment, the exercise information acquiring unit 151 acquires at least one of the exercise intensity, the exercise frequency, and the exercise time period as data representing the exercise pattern on the basis of the measurement data of the activity amount.

By acquiring at least one of the exercise intensity, the exercise frequency, and the exercise time period as data representing the exercise pattern on the basis of the measurement data of the activity amount in the above-described manner, more precise exercise patterns can be acquired.

In the first embodiment, the measurement data of the activity amount includes at least the heart rate measurement data, and the exercise information acquiring unit 151 determines at least one of the exercise intensity, the exercise frequency, and the exercise time period on the basis of the heart rate measurement data and predetermined criteria.

By determining at least one of the exercise intensity, the exercise frequency, and the exercise time period on the basis of the heart rate measurement data and predetermined criteria in the above-described manner, at least one of the exercise intensity, the exercise frequency, and the exercise time period can be acquired through simple arithmetic processing.

The program 141 of the first embodiment causes a computer to execute acquiring the first exercise information related to the exercise performed by the subject person P2 who takes the health guidance before the first guidance report is provided (step S1), acquiring the first diagnosis information including the result of the first health checkup of the subject person P2 (step S2), setting the first weight loss goal of the subject person P2 on the basis of the first diagnosis information (step S3), reading from the storage unit 140 the first trained model 142 that outputs the first advice candidate expected to be valid to achieve the first weight loss goal among a plurality of the first advice candidates based on the first exercise information as the content of the first guidance for the subject person P2 when the first exercise information and the first weight loss goal are input (step S4), and inputting the first exercise information and the first weight loss goal to the first trained model 142, and generating the first guidance report including the content of the first guidance output from the first trained model 142 (step S5).

With the above-described the program 141, the first guidance report including the content of the first guidance that is always effective for the first exercise information and the first weight loss goal of the subject person P2 can be provided to the subject person P2 regardless of the view of the counselor P1.

### Second Embodiment

Next, a second embodiment is described.

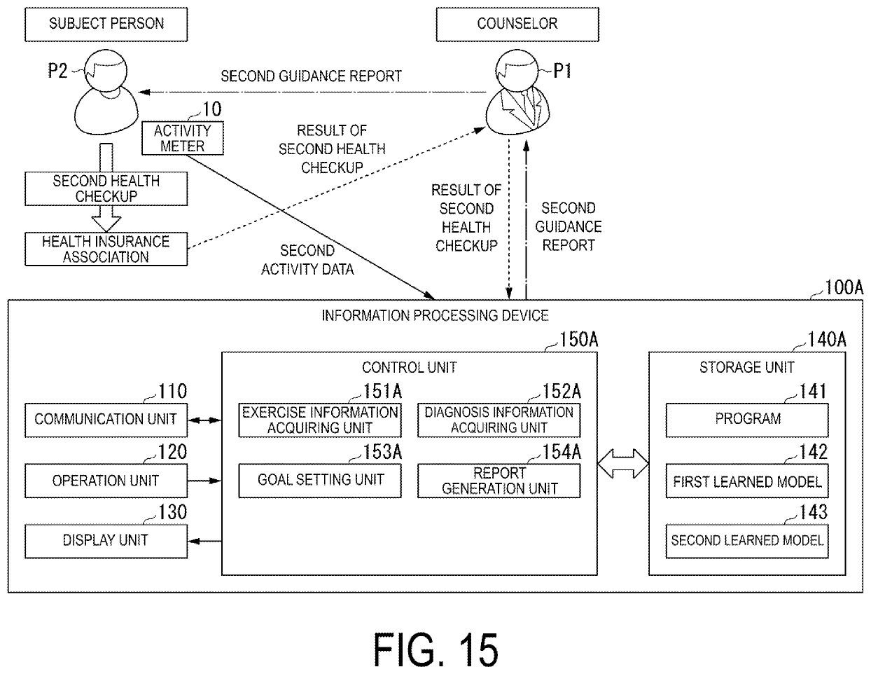

FIG. 15 is a block diagram illustrating a schematic configuration of an information processing device 100A of the second embodiment. In the following description, for the components of the information processing device 100A, the same components as those of the information processing device 100 are denoted with the same reference numerals, and the description thereof is omitted. In addition, in the following description, the counselor is denoted with reference numeral P1, the subject person who takes the health guidance of the counselor P1 is denoted with reference numeral P2, and the activity meter worn on the subject person P2 is denoted with reference numeral 10 for convenience of description as in the first embodiment.

Although the illustration is omitted in FIG. 15, the information processing device 100A is the same as the information processing device 100 at least in that it receives the first activity data from the activity meter 10. As described in the first embodiment, the first activity data includes the measurement data of the activity amount obtained in the measurement period before the first guidance report is provided to the subject person P2, and the identification information of the subject person P2.

As illustrated in FIG. 15, the information processing device 100A differs from the information processing device 100 in that it receives second activity data from the activity meter 10. The second activity data is the measurement data of the activity amount obtained in a period between when the subject person P2 receives the first guidance report and when the subject person P2 takes the second health checkup. After going about his or her daily life while wearing the activity meter 10 for the measurement period specified by the counselor P1 before receiving the second health checkup, the subject person P2 operates the activity meter 10 to transmit the second activity data including the measurement data of the activity amount obtained in the measurement period to the information processing device 100. For example, the second health checkup is a health checkup that the subject person P2 takes before the second meeting with the counselor P1.

When the subject person P2 takes the second health checkup, the result of the second health checkup of the subject person P2 is provided from the health insurance association to the counselor P1. The second embodiment is different from the first embodiment in that the counselor P1 operates the information processing device 100A to input the identification information of the subject person P2 and the result of the second health checkup of the subject person P2 to the information processing device 100A.

Further, the information processing device 100A differs from the information processing device 100 in that it generates a second guidance report including the content of second guidance for the subject person P2 on the basis of the first activity data and the second activity data of the subject person P2, and the result of the second health checkup of the subject person P2 input by the counselor P1.

As in the first embodiment, the information processing device 100A provides a printed material or an electronic file of the generated second guidance report to the counselor P1. After confirming the second guidance report content, the counselor P1 provides the printed material or the electronic file of the second guidance report to the subject person P2.

A configuration of the information processing device 100A is elaborated below. As illustrated in FIG. 15, the information processing device 100A includes the communication unit 110, the operation unit 120, the display unit 130, a storage unit 140A, and a control unit 150A.

The storage unit 140A stores in advance a second trained model 143 in addition to the program 141 and the first trained model 142. Details of the second trained model 143 are described later. The control unit 150A includes an exercise information acquiring unit 151A, a diagnosis information acquiring unit 152A, a goal setting unit 153A, and a report generation unit 154A. These components are functions that are achieved when the control unit 150A executes the guidance report generation program included in the program 141.

As in the first embodiment, the exercise information acquiring unit 151A acquires the first exercise information related to the exercise performed by the subject person P2 who takes the health guidance before receiving the first guidance report on the basis of the first activity data received from the activity meter 10 through the communication unit 110. The exercise information acquiring unit 151A stores the acquired first exercise information in the storage unit 140A.

In addition, on the basis of the second activity data received from the activity meter 10 through the communication unit 110, the exercise information acquiring unit 151A acquires second exercise information related to the exercise performed in a period between when the subject person P2 receives the first guidance report and when the subject person P2 takes the second health checkup. For example, on the basis of the measurement data of the activity amount included in the second activity data, i.e., the measurement data of the activity amount obtained in a period between when the subject person P2 receives the first guidance report and when the subject person P2 takes the second health checkup, the exercise information acquiring unit 151A acquires the exercise pattern of the subject person P2 as the second exercise information. The method for acquiring the exercise pattern is as described in the first embodiment.

The diagnosis information acquiring unit 152A acquires second diagnosis information including the result of the second health checkup of the subject person P2 on the basis of the operation signal output from the operation unit 120. For example, the second diagnosis information includes, as a result of the second health checkup, diagnosis results such as the weight, chest circumference, waist circumference, neutral fat, fasting blood glucose level, blood pressure, and BMI of the subject person P2.

The goal setting unit 153A sets a second weight loss goal of the subject person P2 on the basis of the second diagnosis information. For example, the goal setting unit 153A sets the second weight loss goal such that among the result of the second health checkup of the subject person P2, the diagnosis results of the waist circumference, neutral fat, fasting blood glucose level, and blood pressure are smaller than the diagnosis reference values for metabolic syndrome.

The report generation unit 154A inputs the first exercise information, the second exercise information, and the second weight loss goal to the second trained model 143, and generates the second guidance report including the content of the second guidance output from the second trained model 143. The second trained model 143 is a mathematical model in which when the first exercise information, the second exercise information, and the second weight loss goal are input, a second advice candidate expected to be valid to achieve the second weight loss goal among a plurality of second advice candidates based on the combination of the first exercise information and the second exercise information is output as the content of the second guidance for the subject person P2.

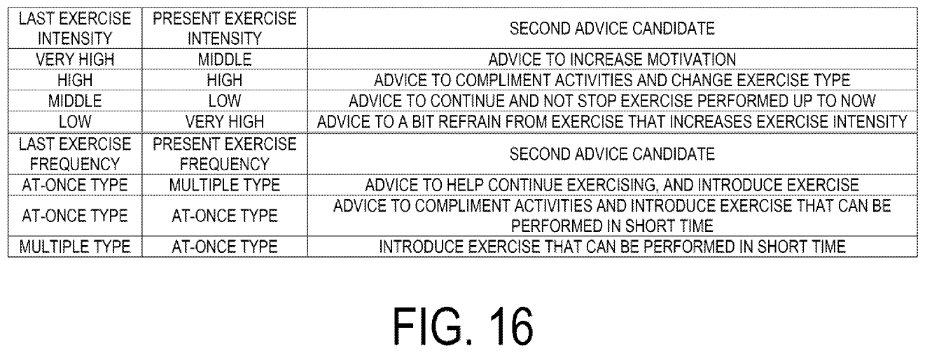

As with the first advice candidate, the second advice candidate includes an advice related to an exercise. FIG. 16 is a diagram illustrating an example of the second advice candidate. As illustrated in FIG. 16, the second advice candidate is an advice based on the combination of the first exercise information and the second exercise information. In FIG. 16, “last exercise intensity” and “last exercise frequency” are the exercise intensity and the exercise frequency included in the first exercise information. In addition, in FIG. 16, “present exercise intensity” and “present exercise frequency” are the exercise intensity and the exercise frequency included in the second exercise information.

As illustrated in FIG. 16, for example, the second advice candidate corresponding to a combination of a last exercise intensity of “very high” and a present exercise intensity of “medium” includes an advice to increase the motivation the subject person P2. The reason for this is that the subject person P2 in this case may have a drastic decrease in motivation.

For example, the second advice candidate corresponding to a combination of a last exercise intensity of “high” and a present exercise intensity of “high” includes an advice for the subject person P2 to change the exercise type while complimenting the activity of the subject person P2. The reason for this is that the subject person P2 in this case does exercises that can ensure the targeted exercise amount, and it is desirable to perform different exercises to avoid getting stuck in a rut.

For example, the second advice candidate corresponding to a combination of a last exercise intensity of “medium” and a present exercise intensity of “low” includes an advice for the subject person P2 to continue and not stop the exercise performed up to now. The reason for this is that the subject person P2 in this case may have a decrease in motivation, hating the exercise itself.

For example, the second advice candidate corresponding to a combination of a last exercise intensity of “low” and a present exercise intensity of “very high” includes an advice for the subject person P2 to refrain from the exercise that increases the exercise intensity. The reason for this is that the subject person P2 in this case has a risk of injury due to the suddenly increased exercise amount.

For example, the second advice candidate corresponding to a combination of a last exercise frequency of “at-once type” and a present exercise frequency of “multiple type” includes an advice to help the subject person P2 continue exercising, and an introduction of an exercise. The reason for this is that the subject person P2 in this case may not know an exercise suitable for performing it on multiple occasions, and it is preferable to give an advice suitable for it.

For example, the second advice candidate corresponding to a combination of a last exercise frequency of “at-once type” and a present exercise frequency of “at-once type” includes an advice complimenting the activity of the subject person P2, and an introduction of an exercise that can be performed in a short time. The reason for this is that for the subject person P2 in this case, it is preferable to compliment the activities so that the subject person P2 maintains motivation and continues the exercise in the future. In addition, the subject person P2 in this case may not have much time to devote to the exercise, and it is therefore preferable to introduce the exercise that can be performed in a short time.

For example, the second advice candidate corresponding to a combination of a last exercise frequency of “multiple type” and a present exercise frequency of “at-once type” includes an introduction of an exercise that can be performed in a short time. The reason for this is that the subject person P2 in this case may be busy in personal life and may not be able to devote time to the exercise, and it is therefore preferable to introduce an exercise that can be efficiently performed in a short time.

Note that as with the first advice candidate, the second advice candidate may include not only the above-described advices related to the exercise but also an advice related to diet and sleep. In addition, the second guidance report may include other information such as the result of the first health checkup and the weight loss goal in addition to the content of the second guidance output from the second trained model 143.

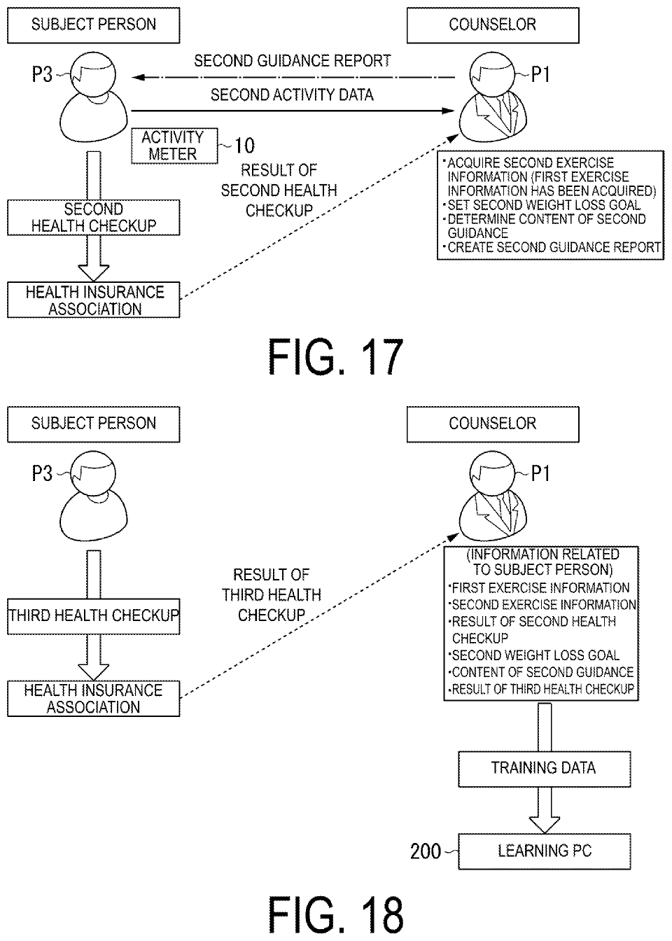

As with the first trained model 142, the second trained model 143 is a mathematical model generated through machine learning such as deep learning, for example. In the following description, a procedure of generating the second trained model 143 is described with reference to FIGS. 17 and 18.

As illustrated in FIG. 17, after going about his or her daily life while wearing the activity meter 10 for the measurement period specified by the counselor P1 before receiving the second health checkup, the subject person P3 different from the subject person P2 provides the second activity data obtained by the activity meter 10 to the counselor P1 and takes the second health checkup. When the subject person P3 takes the second health checkup, the result of the second health checkup of the subject person P3 is provided from the health insurance association to the counselor P1.

On the basis of the second activity data received from the subject person P3, the counselor P1 acquires the second exercise information of the subject person P3. Note that at this point of time, the counselor P1 has acquired the first exercise information of the subject person P2 as described in the first embodiment. In addition, on the basis of the result of the second health checkup provided from the health insurance association, the counselor P1 sets the second weight loss goal of the subject person P3.

Then, on the basis of the combination of the first exercise information and the second exercise information of the subject person P3, the counselor P1 determines the second advice candidate for achieving the second weight loss goal among the plurality of second advice candidates as those illustrated in FIG. 16 as the content of the second guidance for the subject person P3. Here, the counselor P1 may not necessarily determine the content of the second guidance completely matching the correspondence relationship of the second advice candidate and the combination of the first exercise information and the second exercise information as that illustrated in FIG. 16.

For example, even for a combination of a last exercise intensity of “very high” and a present exercise intensity of “medium”, the counselor P1 may determine the second advice candidate corresponding to a different combination as the content of the second guidance in consideration of the second weight loss goal and the like. Alternatively, the counselor P1 may determine the second advice candidate corresponding to a combination of a last exercise intensity of “very high” and a present exercise intensity of “medium” as the content of the second guidance in accordance with the relationship illustrated in FIG. 16. In this manner, the relationship illustrated in FIG. 16 is used for reference only, and a different content of the second guidance may be determined for the same combination of the first exercise information and the second exercise information in accordance with the second weight loss goal.

The counselor P1 creates the second guidance report including the content of the second guidance determined in the above-described manner, the result of the second health checkup of the subject person P3, and the second weight loss goal. The counselor P1 provides the created second guidance report to the subject person P3.

As illustrated in FIG. 18, after going about his or her daily life in accordance with the second guidance report received from the counselor P1, the subject person P3 takes a third health checkup. For example, the third health checkup is a health checkup that the subject person P3 takes after receiving the second guidance report. When the subject person P3 takes the third health checkup, the result of the third health checkup of the subject person P3 is provided from the health insurance association to the counselor P1.

On the basis of the result of the third health checkup of the subject person P3, the counselor P1 confirms whether the second weight loss goal of the subject person P3 has been achieved by the content of the second guidance in the second guidance report. Specifically, the counselor P1 confirms whether the diagnosis results of the waist circumference, neutral fat, fasting blood glucose level, and blood pressure of the subject person P3 are smaller than the diagnosis reference values for metabolic syndrome among the results of the third health checkup.

In this manner, the counselor P1 acquires, as the information related to the subject person P3, the first exercise information, the second exercise information, the result of the second health checkup, the second weight loss goal, the content of the second guidance, and the result of the third health checkup. The result of the third health checkup is information representing whether the second weight loss goal of the subject person P3 has been achieved by the content of the second guidance in the second guidance report.

The counselor P1 acquires the first exercise information, the second exercise information, and the second weight loss goal as data to be input, i.e., learning data from among information related to the subject person P3. In addition, the counselor P1 acquires the content of the second guidance that has succeeded in achieving the second weight loss goal of the subject person P3 as data to be output, i.e., correct data from among the information related to the subject person P3.

The counselor P1 acquires the above-described set of the learning data and the correct data as training data, and stores the acquired training data in linkage with the identification information of the subject person P3 in a learning PC 200. The learning PC 200 may be the same PC as the information processing device 100A, or a PC different from the information processing device 100A. Note that in the case where the second weight loss goal of the subject person P3 has not been successfully achieved, the counselor P1 may not acquire the training data related to the subject person P3.

The same procedure as the above-mentioned procedure is sequentially applied to the subject person other than the subject person P3, and the training data linked with the identification information of the subject person other than the subject person P3 is sequentially stored in the learning PC 200. When the number of pieces of the training data stored in the learning PC 200 reaches a predetermined number, the counselor P1 operates the learning PC 200 such that supervised learning is performed on the basis of the stored training data.

As a result, a trained model is generated in which, when data including the first exercise information, the second exercise information, and the second weight loss goal of an unknown subject person is input, correct data corresponding to the input data, i.e., the second advice candidate expected to be valid to achieve the second weight loss goal of the unknown subject person among the plurality of second advice candidates as those illustrated in FIG. 16, is output as the content of the second guidance for the unknown subject person.

The trained model generated by the learning PC 200 in this manner is the second trained model 143 of the second embodiment. Specifically, the second trained model 143 is a mathematical model in which when the first exercise information, the second exercise information and the second weight loss goal of the subject person P2 as a unknown subject person are input, the second advice candidate expected to be valid to achieve the second weight loss goal of the subject person P2 among the plurality of second advice candidates based on the combination of the first exercise information and the second exercise information is output as the content of the second guidance for the subject person P2.

Next, an operation of the information processing device 100A having the above-mentioned configuration is described.

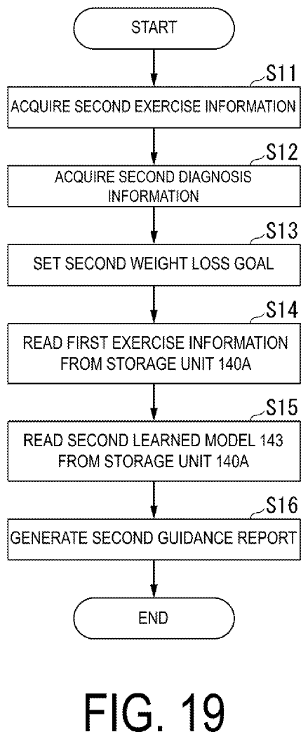

FIG. 19 is a flowchart illustrating a guidance report generation process executed by the control unit 150A. When the control unit 150A detects a reception of an operation of instructing generation of the second guidance report from the counselor P1 on the basis of the operation signal output from the operation unit 120, the control unit 150A reads the guidance report generation program from the storage unit 140A and executes the program to execute the guidance report generation process illustrated in FIG. 19.

As illustrated in FIG. 19, when the guidance report generation process is started, first, the control unit 150A acquires the second exercise information related to the exercise performed in a period between when the subject person P2 receives the first guidance report and when the subject person P2 takes the second health checkup on the basis of the second activity data received from the activity meter 10 through the communication unit 110 (step S11). Note that before the guidance report generation process illustrated in FIG. 19 is started, the storage unit 140A has stored the first exercise information of the subject person P2.

For example, at step S11, the control unit 150A acquires the exercise pattern of the subject person P2 as the second exercise information on the basis of the measurement data of the activity amount included in the second activity data, i.e., the measurement data of the activity amount obtained in a period between when the subject person P2 receives the first guidance report and when the subject person P2 takes the second health checkup. The process of this step S11 is the same process as the process executed by the exercise information acquiring unit 151A, and therefore the description related to step S11 is omitted.

Subsequently, the control unit 150A acquires the second diagnosis information including the result of the second health checkup of the subject person P2 on the basis of the operation signal output from the operation unit 120 (step S12). For example, the second diagnosis information includes, as a result of the second health checkup, diagnosis results such as the weight, chest circumference, waist circumference, neutral fat, fasting blood glucose level, blood pressure (systolic blood pressure and diastolic blood pressure), and BMI of the subject person P2. The process of this step S12 is the same process as the process executed by the diagnosis information acquiring unit 152A, and therefore the description related to step S12 is omitted.

Subsequently, the control unit 150A sets the second weight loss goal of the subject person P2 on the basis of the second diagnosis information (step S13). For example, at step S13, the control unit 150A sets the second weight loss goal such that the diagnosis results of the waist circumference, neutral fat, fasting blood glucose level, and blood pressure included in the result of the second health checkup of the subject person P2 are smaller than the diagnosis reference values for metabolic syndrome. The process of this step S13 is the same process as the process executed by the goal setting unit 153A, and therefore the description related to step S13 is omitted.

Subsequently, the control unit 150A reads the first exercise information from the storage unit 140A (step S14). In addition, the control unit 150A reads the second trained model 143 from the storage unit 140A (step S15).

Then, the control unit 150A inputs the first exercise information, the second exercise information, and the second weight loss goal to the second trained model 143, and generates the second guidance report including the content of the second guidance output from the second trained model 143 (step S16). The control unit 150A may generate the second guidance report including other information such as the result of the second health checkup and the second weight loss goal in addition to the content of the second guidance output from the second trained model 143. The process of this step S16 is the same process as the process executed by the report generation unit 154A, and therefore the description related to step S16 is omitted.

### Effects of Second Embodiment

As described above, in the information processing device 100A of the second embodiment, the exercise information acquiring unit 151A acquires the second exercise information related to the exercise performed in a period between when the subject person P2 receives the first guidance report and when the subject person P2 takes the second health checkup, the diagnosis information acquiring unit 152A acquires the second diagnosis information including the result of the second health checkup of the subject person P2, the goal setting unit 153A sets the second weight loss goal of the subject person P2 on the basis of the second diagnosis information, the storage unit 140A stores in advance the second trained model 143 in which when the first exercise information, the second exercise information, and the second weight loss goal are input, the second advice candidate expected to be valid to achieve the second weight loss goal among the plurality of second advice candidates based on the combination of the first exercise information and the second exercise information is output, as the content of the second guidance for the subject person P2, and the report generation unit 154A inputs the first exercise information, the second exercise information, and the second weight loss goal the second trained model 143, and generates the second guidance report including the content of the second guidance output from the second trained model 143.

As described above for the explanation of the generation procedure for the second trained model 143, when the counselor P1 creates the second guidance report, the content of the second guidance provided to the subject person may differ depending on the view of the counselor P1 even for the same combination of the exercise pattern. Such a content of the second guidance dependent on the view of the counselor P1 may or may not be effective for the subject person depending on the case.

On the other hand, the information processing device 100A of the second embodiment inputs the first exercise information, the second exercise information and the second weight loss goal of the subject person P2 to the second trained model 143, and generates the second guidance report including the content of the second guidance output from the second trained model 143. The second trained model 143 is a mathematical model generated through machine learning in which when the first exercise information, the second exercise information and the second weight loss goal of the subject person P2 are input, the second advice candidate expected to be valid to achieve the second weight loss goal among the plurality of second advice candidates based on the combination of the first exercise information and the second exercise information is output as the content of the second guidance for the subject person P2. Thus, regardless of the first exercise information, the second exercise information and the second weight loss goal input to the second trained model 143, the correct content of the second guidance, i.e., the content of the second guidance that has succeeded in achieving the second weight loss goal in the past history is always output from the second trained model 143.

Therefore, according to the information processing device 100A of the second embodiment, the second guidance report including the content of the second guidance that is always effective for the first exercise information, the second exercise information and the second weight loss goal of the subject person P2 can be provided to the subject person P2 regardless of the view of the counselor P1.

### Third Embodiment

Next, a third embodiment is described.

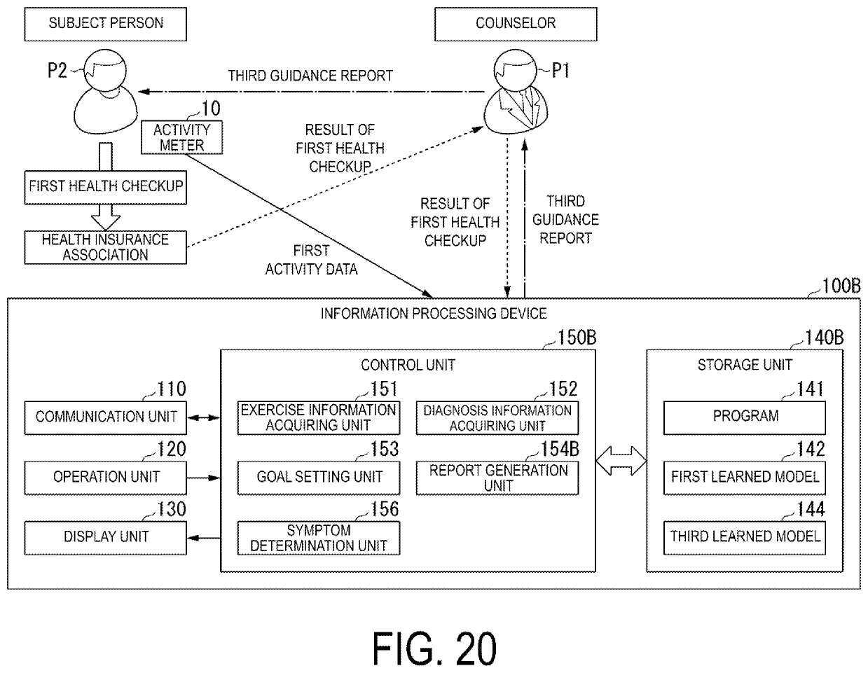

FIG. 20 is a block diagram illustrating a schematic configuration of an information processing device 100B of the third embodiment. In the following description, for the components of the information processing device 100B, the same components as those of the information processing device 100 are denoted with the same reference numerals, and the description thereof is omitted. In addition, in the following description, the counselor is denoted with reference numeral P1, the subject person who takes the health guidance of the counselor P1 is denoted with reference numeral P2, and the activity meter worn on the subject person P2 is denoted with reference numeral 10 for convenience of description as in the first embodiment.

The information processing device 100B differs from the information processing device 100 in that it generates a third guidance report including a content of third guidance for the subject person P2 on the basis of the first activity data of the subject person P2 received from the activity meter 10 and the result of the first health checkup of the subject person P2 input by the counselor P1. As in the first embodiment, the first activity data transmitted from the activity meter 10 to the information processing device 100B includes the measurement data of the activity amount obtained in the measurement period before the first guidance report is provided to the subject person P2.

As in the first embodiment, the information processing device 100B provides a printed material or an electronic file of the generated third guidance report to the counselor P1. After confirming the content of the third guidance report, the counselor P1 provides the printed material or the electronic file of the third guidance report to the subject person P2.

In the following description, a configuration of the information processing device 100B is elaborated. As illustrated in FIG. 20, the information processing device 100B includes the communication unit 110, the operation unit 120, the display unit 130, a storage unit 140B, and a control unit 150B.

The storage unit 140B stores a third trained model 144 in advance in addition to the program 141 and the first trained model 142. Details of the third trained model 144 are described later. The control unit 150B includes the exercise information acquiring unit 151, the diagnosis information acquiring unit 152, the goal setting unit 153, a report generation unit 154B, and a symptom determination unit 156. These components are functions that are achieved when the control unit 150B executes the guidance report generation program included in the program 141.

The symptom determination unit 156 determines whether the obesity symptom of the subject person P2 is severe or mild on the basis of the first diagnosis information acquired by the diagnosis information acquiring unit 152. As described in first embodiment, the first diagnosis information includes the BMI (Body Mass Index) of the subject person P2 as a result of the first health checkup of the subject person P2. The symptom determination unit 156 determines that the obesity symptom of the subject person P2 is severe when the BMI of the subject person P2 is a predetermined value or greater, and determines that the obesity symptom of the subject person P2 is mild when the BMI of the subject person P2 is smaller than a predetermined value.

For example, according to the criteria of The Japan Society for the Study of Obesity, a BMI of 25 or more but less than 30 is obesity level 1, a BMI of 30 or more but less than 35 is obesity level 2, a BMI of 35 or more but less than 40 is obesity level 3, and a BMI of 40 or more is obesity level 4. In view of this, the predetermined value may be set to 35. In this case, the symptom determination unit 156 determines that the obesity symptom of the subject person P2 is severe when the BMI of the subject person P2 is 35 or greater, and determines that the obesity symptom of the subject person P2 is mild when the BMI of the subject person P2 is smaller than 35.

The report generation unit 154B inputs the first exercise information, the determination result of the obesity symptom, and the first weight loss goal to the third trained model 144, and generates the third guidance report including the content of the third guidance output from the third trained model 144. The third trained model 144 is a mathematical model in which when the first exercise information, the determination result of the obesity symptom, and the first weight loss goal are input, the third advice candidate expected to be valid to achieve the first weight loss goal among a plurality of third advice candidates based on the combination of the first exercise information and the determination result of the obesity symptom is output, as the content of the third guidance for the subject person P2.

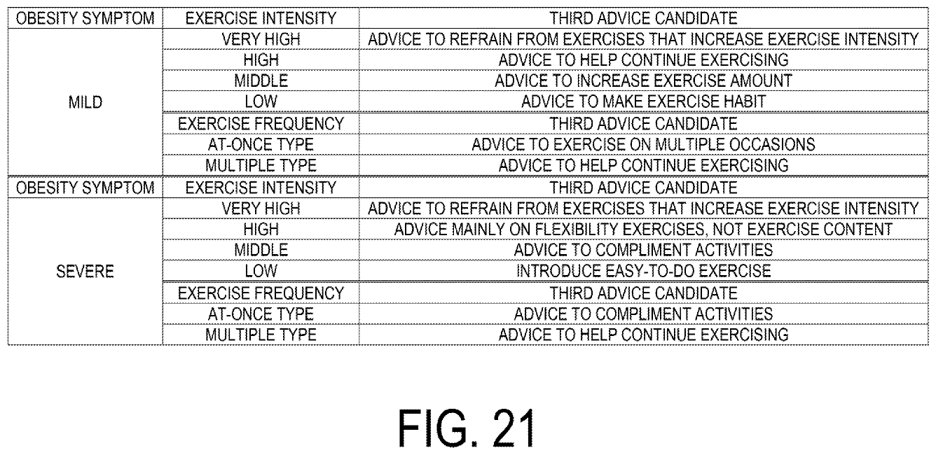

As with the first advice candidate, the third advice candidate is an advice related to an exercise. FIG. 21 is a diagram illustrating an example of the third advice candidate. As illustrated in FIG. 21, the third advice candidate is an advice based on the combination of the first exercise information and the determination result of the obesity symptom. In the third advice candidate, basically, the advice for the subject person whose obesity symptom is severe is easier than the advice for the subject person whose obesity symptom is mild. The subject person whose obesity symptom is severe has a greater burden of the exercise than that of the subject person whose obesity symptom is mild. In addition, since the targeted numerical value is high, advices for maintaining motivation to exercise for longer periods of time are mainly used.

For example, in the case where the obesity symptom is mild, the third advice candidate corresponding to the exercise intensity of “very high” includes an advice for the subject person P2 to refrain from exercises that increase the exercise intensity. The reason for this is that the subject person P2 subjected to the health guidance has not exercised much in the daily life before the health guidance period, and the risk of injury is high if the subject person P2 suddenly performs very high-intensity exercises.

For example, in the case where the obesity symptom is mild, the third advice candidate corresponding to the exercise intensity of “high” includes an advice to help the subject person P2 continue exercising. The reason for this is that the subject person P2 in this case has already performed the exercise that can ensure the targeted exercise amount in the daily life.

For example, in the case where the obesity symptom is mild, the third advice candidate corresponding to the exercise intensity of “medium” includes an advice to increase the exercise amount of the subject person P2. The reason for this is that the subject person P2 in this case does not perform the exercise that can ensure the targeted exercise amount in the daily life.

For example, in the case where the obesity symptom is mild, the third advice candidate corresponding to the exercise intensity of “low” includes an advice for the subject person P2 to make exercise a habit. The reason for this is that the subject person P2 in this case may not be able to continue and follow the advice for a long time even if the subject person P2 receives the advice to increase the exercise intensity, and needs to make exercise a habit first.

For example, in the case where the obesity symptom is mild, the third advice candidate corresponding to the exercise frequency of “at-once type” includes an advice for the subject person P2 to perform the exercise on multiple occasions. The reason for this is that the subject person P2 in this case is unbalanced between the exercise amount on days when exercise is performed and the exercise amount on days when exercise is not performed in the daily life.

For example, in the case where the obesity symptom is mild, the third advice candidate corresponding to the exercise frequency of “multiple type” includes an advice to help the subject person P2 continue exercising. The reason for this is that the subject person P2 in this case can easily maintain the exercise amount because, even when the exercise is not performed in a certain time period, the exercise is performed in other time periods in the daily life.

For example, in the case where the obesity symptom is severe, the third advice candidate corresponding to the exercise intensity of “very high” includes an advice for the subject person P2 to refrain from exercises that increase the exercise intensity. The reason for this is that the subject person P2 in this case is not in the habit of exercising in the daily life before the health guidance period, and therefore the risk of injury is high if the subject person P2 suddenly performs very high-intensity exercises.

For example, in the case where the obesity symptom is severe, the third advice candidate corresponding to the exercise intensity of “high” includes an advice mainly on flexibility exercises, not the exercise content. The reason for this is that the subject person P2 in this case has a greater burden on the body than that of the subject person whose obesity symptom is mild, and the risk of injury of the subject person P2 should be reduced as much as possible.

For example, in the case where the obesity symptom is severe, the third advice candidate corresponding to the exercise intensity of “medium” includes an advice complimenting the activity of the subject person P2. The reason for this is that the subject person P2 in this case has performed an activity with a considerably greater burden for the body type than that of the subject person whose obesity symptom is mild even though the exercise intensity is “medium”, and it is therefore necessary to maintain the motivation of the subject person P2.

For example, in the case where the obesity symptom is severe, the third advice candidate corresponding to the exercise intensity of “low” includes an introduction of an easy-to-do exercise to the subject person P2. The reason for this is that the subject person P2 in this case does not know what kind of exercise to do, and may not be able to continue and follow the advice for a long time even if the subject person P2 receives the advice to increase the exercise intensity, and therefore, needs to make exercise a habit first.

For example, in the case where the obesity symptom is severe, the third advice candidate corresponding to the exercise frequency of “at-once type” includes an advice complimenting the activity of the subject person P2. The reason for this is that the subject person P2 in this case needs to maintain the motivation by complimenting the activity.

For example, in the case where the obesity symptom is severe, the third advice candidate corresponding to the exercise frequency of “multiple type” includes an advice to help the subject person P2 continue exercising. The reason for this is that the subject person P2 in this case can easily maintain the exercise amount because, even when the exercise is not performed in a certain time period, the exercise is performed in other time periods in the daily life.

Note that as with the first advice candidate, the third advice candidate may include not only the above-described advices related to the exercise but also an advice related to diet and sleep. In addition, the third guidance report may include other information such as the result of the first health checkup and the first weight loss goal in addition to the content of the third guidance output from the third trained model 144.

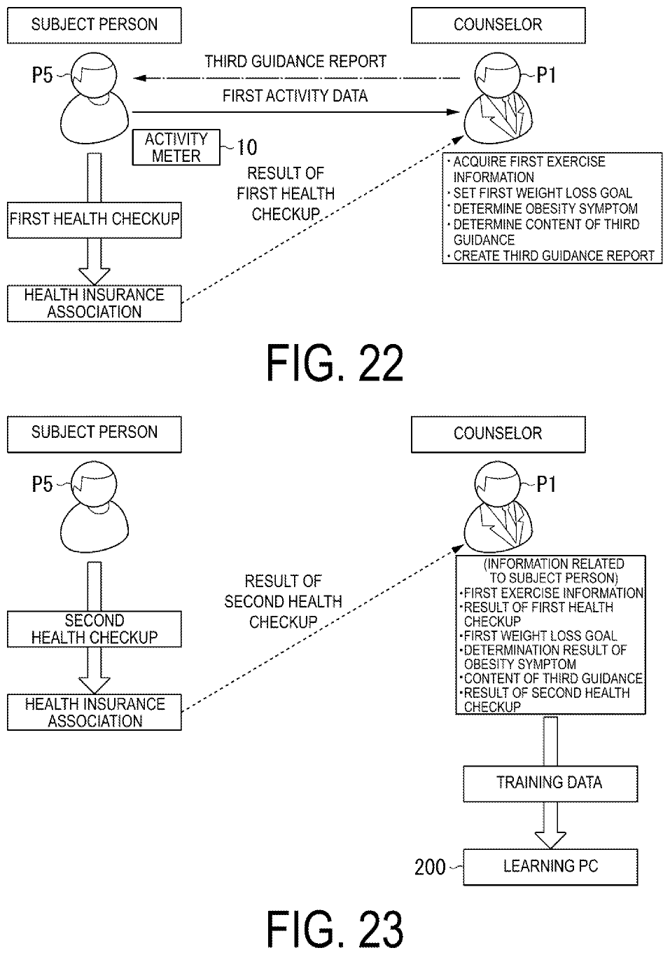

As with the first trained model 142, the third trained model 144 is a mathematical model generated through machine learning such as deep learning, for example. In the following description, a procedure of generating the third trained model 144 is described with reference to FIGS. 22 and 23.

As illustrated in FIG. 22, before the first guidance report is provided, a subject person P5 different from the subject person P2 provides the first activity data obtained by the activity meter 10 to the counselor P1 after going about his or her daily life while wearing the activity meter 10 for the measurement period specified by the counselor P1, and takes the first health checkup. When the subject person P5 takes the first health checkup, the result of the first health checkup of the subject person P5 is provided from the health insurance association to the counselor P1.

The counselor P1 acquires the first exercise information of the subject person P5 on the basis of the first activity data received from the subject person P5. In addition, the counselor P1 sets the first weight loss goal of the subject person P5 on the basis of the result of the first health checkup provided from the health insurance association, and determines whether the obesity symptom of the subject person P5 is severe or mild. For example, the counselor P1 determines that the obesity symptom of the subject person P5 is severe when the BMI of the subject person P5 is 35 or greater, and determines that the obesity symptom of the subject person P5 is mild when the BMI of the subject person P5 is smaller than 35.

Then, on the basis of the combination of the exercise pattern and the obesity symptom of the subject person P5, the counselor P1 determines the first weight loss goal for achieving the third advice candidate among the plurality of third advice candidates as those illustrated in FIG. 21 as the content of the third guidance for the subject person P5. Here, the counselor P1 may not necessarily determine the content of the third guidance completely matching the correspondence relationship of the third advice candidate and the combination of the exercise pattern and the obesity symptom illustrated in FIG. 21.

For example, even in the case where the exercise intensity of the subject person P5 is “high”, the counselor P1 may determine the third advice candidate corresponding to the exercise intensity of “very high” as the content of the third guidance in consideration of the first weight loss goal and the like. Alternatively, in the case where the exercise intensity of the subject person P5 is “high”, the counselor P1 may determine the third advice candidate corresponding to the exercise intensity of “high” as the content of the third guidance in accordance with the relationship illustrated in FIG. 21 in consideration of the first weight loss goal and the like. As described above, the relationship illustrated in FIG. 21 is used for reference only, and different contents of the third guidance may be determined for the same combination of the exercise pattern and the obesity symptom in accordance with the first weight loss goal of the subject person P5.

The counselor P1 creates the third guidance report including the content of the third guidance determined in the above-described manner, the result of the first health checkup of the subject person P5, and the first weight loss goal. The counselor P1 provides the created third guidance report to the subject person P5.

As illustrated in FIG. 23, after going about his or her daily life in accordance with the third guidance report received from the counselor P1, the subject person P5 takes the second health checkup. For example, the second health checkup is a health checkup that the subject person P5 takes first after receiving the third guidance report. When the subject person P5 takes the second health checkup, the result of the second health checkup of the subject person P5 is provided from the health insurance association to the counselor P1.

On the basis of the result of the second health checkup of the subject person P5, the counselor P1 confirms whether the first weight loss goal of the subject person P5 has been achieved by the content of the third guidance in the third guidance report. Specifically, the counselor P1 confirms whether the diagnosis results of the waist circumference, neutral fat, fasting blood glucose level, and blood pressure of the subject person P5 among the result of the second health checkup are smaller than the diagnosis reference values for metabolic syndrome.

In this manner, the counselor P1 acquires, as information related to the subject person P5, the first exercise information, the result of the first health checkup, the first weight loss goal, the determination result of the obesity symptom, the content of the third guidance, and the result of the second health checkup. The result of the second health checkup is information representing whether the first weight loss goal of the subject person P5 has been achieved by the content of the third guidance in the third guidance report.

The counselor P1 acquires the first exercise information, the determination result of the obesity symptom, and the first weight loss goal as data to be input, i.e., learning data from among information related to the subject person P5. In addition, the counselor P1 acquires the content of the third guidance that has succeeded in achieving the first weight loss goal of the subject person P5 as data to be output, i.e., correct data from among the information related to the subject person P5.

The counselor P1 acquires the above-described set of the learning data and the correct data as training data, and stores the acquired training data in linkage with the identification information of the subject person P5 in the learning PC 200. The learning PC 200 may be the same PC as the information processing device 100B, or a PC different from the information processing device 100B. Note that when the first weight loss goal of the subject person P5 has not been successfully achieved, the counselor P1 may not acquire the training data related to the subject person P5.

The same procedure as the above-mentioned procedure is sequentially applied to the subject person other than the subject person P5, and the training data linked with the identification information of the subject person other than the subject person P5 is sequentially stored in the learning PC 200. When the number of pieces of the training data stored in the learning PC 200 reaches a predetermined number, the counselor P1 operates the learning PC 200 such that supervised learning is performed on the basis of the stored training data.

As a result, a trained model is generated in which when data including the exercise pattern, the determination result of the obesity symptom, and the first weight loss goal of an unknown subject person is input, correct data corresponding to the input data, i.e., the third advice candidate expected to be valid to achieve the first weight loss goal of the unknown subject person among the plurality of third advice candidates as those illustrated in FIG. 21, is output as the content of the third guidance for the unknown subject person.

The trained model generated by the learning PC 200 in this manner is the third trained model 144 of the third embodiment. Specifically, the third trained model 144 is a mathematical model in which when the first exercise information, the determination result of the obesity symptom and the first weight loss goal of the subject person P2 as an unknown subject person are input, the third advice candidate expected to be valid to achieve the first weight loss goal of the subject person P2 among the plurality of the third advice candidates based on the combination of the first exercise information and the determination result of the obesity symptom is output as the content of the third guidance for the subject person P2.

Next, an operation of the information processing device 100B having the above-mentioned configuration is described.

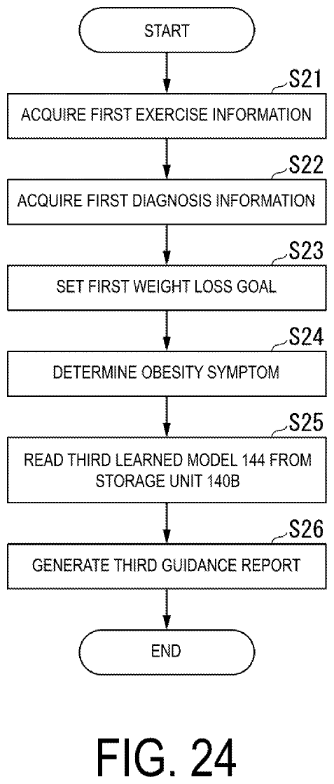

FIG. 24 is a flowchart illustrating a guidance report generation process executed by the control unit 150B. When the control unit 150B detects a reception of an operation of instructing generation of the third guidance report from the counselor P1 on the basis of the operation signal output from the operation unit 120, the control unit 150B reads the guidance report generation program from the storage unit 140B and executes the program to execute the guidance report generation process illustrated in FIG. 24.

As illustrated in FIG. 24, when the guidance report generation process is started, first, the control unit 150B acquires the first exercise information related to the exercise performed by the subject person P2 who takes the health guidance before the first guidance report is provided on the basis of the first activity data received from the activity meter 10 through the communication unit 110 (step S21).

For example, at step S21, on the basis of the measurement data of the activity amount included in the first activity data, i.e., the measurement data of the activity amount obtained in the measurement period before the first guidance report is provided to the subject person P2, the control unit 150B acquires the exercise pattern of the subject person P2 as the first exercise information. The process of this step S21 is the same process as the process of step S1 illustrated in FIG. 14, and therefore the description related to step S21 is omitted.

Subsequently, the control unit 150B acquires the first diagnosis information including the result of the first health checkup of the subject person P2 on the basis of the operation signal output from the operation unit 120 (step S22). For example, the first diagnosis information includes, as a result of the first health checkup, diagnosis results such as the weight, chest circumference, waist circumference, neutral fat, fasting blood glucose level, blood pressure (systolic blood pressure and diastolic blood pressure), and BMI of the subject person P2. The process of this step S22 is the same process as the process of step S2 illustrated in FIG. 14, and therefore the description related to step S22 is omitted.

Subsequently, the control unit 150B sets the first weight loss goal of the subject person P2 on the basis of the first diagnosis information (step S23). For example, at step S23, the control unit 150B sets the first weight loss goal such that the diagnosis results of the waist circumference, neutral fat, fasting blood glucose level, and blood pressure of the subject person P2 are smaller than the diagnosis reference values for metabolic syndrome. The process of this step S23 is the same process as the process of step S3 illustrated in FIG. 14, and therefore the description related to step S23 is omitted.

Subsequently, the control unit 150B determines whether the obesity symptom of the subject person P2 is severe or mild on the basis of the first diagnosis information (step S24). For example, at step S24, the control unit 150B determines that the obesity symptom of the subject person P2 is severe when the BMI of the subject person P2 is a predetermined value or greater, and determines that the obesity symptom of the subject person P2 is mild when the BMI of the subject person P2 is smaller than a predetermined value. The process of this step S24 is the same process as the process executed by the symptom determination unit 156, and therefore the description related to step S24 is omitted.

Subsequently, the control unit 150B reads the third trained model 144 from the storage unit 140B (step S25). Then, the control unit 150B inputs the first exercise information, the determination result of the obesity symptom, and the first weight loss goal to the third trained model 144, and generates the third guidance report including the content of the third guidance output from the third trained model 144 (step S26). The control unit 150B may generate the third guidance report including other information such as the result of the first health checkup and the first weight loss goal in addition to the content of the third guidance output from the third trained model 144. The process of this step S26 is the same process as the process executed by the report generation unit 154B, and therefore the description related to step S26 is omitted.

### Effects of Third Embodiment

As described above, the information processing device 100B of the third embodiment further includes the symptom determination unit 156 that determines whether the obesity symptom of the subject person P2 is severe or mild on the basis of the first diagnosis information, the storage unit 140B stores in advance the third trained model 144 in which when the first exercise information, the determination result of the obesity symptom, and the first weight loss goal are input, the third advice candidate expected to be valid to achieve the first weight loss goal among the plurality of the third advice candidates based on the combination of the first exercise information and the determination result of the obesity symptom is output as the content of the third guidance for the subject person P2, and the report generation unit 154B inputs the first exercise information, the determination result of the obesity symptom, and the first weight loss goal to the third trained model 144, and generates the third guidance report including the content of the third guidance output from the third trained model 144.

As described above for the explanation of the generation procedure for the third trained model 144, when the counselor P1 creates the third guidance report, the content of the third guidance provided to the subject person may differ depending on the view of the counselor P1 even for the same combination of the exercise pattern and the obesity symptom. Such a content of the third guidance dependent on the view of the counselor P1 may or may not be effective for the subject person depending on the case.

On the other hand, the information processing device 100B of the third embodiment inputs the first exercise information, the determination result of the obesity symptom and the first weight loss goal of the subject person P2 to the third trained model 144, and generates the third guidance report including the content of the third guidance output from the third trained model 144. The third trained model 144 is a mathematical model generated through machine learning in which when the first exercise information, the determination result of the obesity symptom and the first weight loss goal of the subject person P2 are input, the third advice candidate expected to be valid to achieve the first weight loss goal among the plurality of the third advice candidates based on the combination of the first exercise information and the determination result of the obesity symptom is output as the content of the third guidance for the subject person P2. Thus, regardless of the first exercise information, the determination result of the obesity symptom and the first weight loss goal input to the third trained model 144, the correct content of the third guidance, i.e., the content of the third guidance that has succeeded in achieving the first weight loss goal in the past history is always output from the third trained model 144.

Therefore, with the information processing device 100B of the third embodiment, the third guidance report including the content of the third guidance that is always effective for the first exercise information, the determination result of the obesity symptom and the first weight loss goal of the subject person P2 can be provided to the subject person P2 regardless of the view of the counselor P1.

In the third embodiment, the first diagnosis information includes, as a result of the first health checkup, the BMI (Body Mass Index) of the subject person P2, and the symptom determination unit 156 determines that the obesity symptom is severe when the BMI is a predetermined value or greater, and determines that the obesity symptom is mild when the BMI is smaller than a predetermined value.

As described above, by determining that the obesity symptom is severe when the BMI of the subject person P2 is a predetermined value or greater, and determining that the obesity symptom is mild when the BMI is smaller than a predetermined value, the obesity symptom of the subject person P2 can be determined through a simple process.

The technical scope of the present disclosure is not limited to the above embodiments, and various changes can be made without departing from the gist of the present disclosure.

For example, the above-mentioned embodiment describes an example in which the exercise information acquiring unit 151 acquires the exercise pattern of the subject person P2 as the first exercise information on the basis of the measurement data of the activity amount included in the first activity data. As another example, it is possible to adopt an exercise information acquiring unit that acquires the measurement data of the activity amount included in the first activity data as the first exercise information of the subject person P2.

### Overview of Present Disclosure

An overview of the present disclosure is described below.

### Supplementary Note 1

An information processing device includes an exercise information acquiring unit configured to acquire first exercise information related to an exercise performed by a subject person who receives health guidance, a diagnosis information acquiring unit configured to acquire first diagnosis information including a result of a first health checkup of the subject person, a goal setting unit configured to set a first weight loss goal of the subject person based on the first diagnosis information, a storage unit configured to store in advance a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input, and a report generation unit configured to input the first exercise information and the first weight loss goal to the first trained model, and generate a first guidance report including the content of the first guidance output from the first trained model.

As described above, the information processing device according to supplementary note 1 inputs the first exercise information and the first weight loss goal of the subject person to the first trained model, and generates the first guidance report including the content of the first guidance output from the first trained model. The first trained model is a mathematical model generated through machine learning in which when the first advice candidate expected to be valid to achieve the first weight loss goal among a plurality of the first advice candidates based on the first exercise information is output as the content of the first guidance for the subject person when the first exercise information and the first weight loss goal of the subject person are input. Thus, regardless of the first exercise information and the first weight loss goal input to the first trained model, the correct content of the first guidance, i.e., the content of the first guidance that has succeeded in achieving the first weight loss goal in the past history is always output from the first trained model.

Thus, according to the information processing device according to supplementary note 1, the first guidance report including the content of the first guidance that is always effective for the first exercise information and the first weight loss goal of the subject person can be provided to the subject person regardless of the view of the counselor.

### Supplementary Note 2

The information processing device according to supplementary note 1, in which the first diagnosis information includes, as a result of the first health checkup, diagnosis results of a waist circumference, a neutral fat, a fasting blood glucose level, and a blood pressure of the subject person, and the goal setting unit sets the first weight loss goal such that the diagnosis results of the waist circumference, the neutral fat, the fasting blood glucose level, and the blood pressure are smaller than diagnosis reference values for metabolic syndrome.

According to the information processing device according to supplementary note 2, by setting the first weight loss goal such that the diagnosis results of the waist circumference, neutral fat, fasting blood glucose level, and blood pressure are smaller than the diagnosis reference values for metabolic syndrome, the first weight loss goal of the subject person can be correctly set.

### Supplementary Note 3

The information processing device according to supplementary note 1 or 2, in which the exercise information acquiring unit acquires an exercise pattern of the subject person as the first exercise information based on measurement data of an activity amount obtained from a measurement device worn on the subject person.

According to the information processing device according to supplementary note 3, by acquiring the exercise pattern of the subject person as the first exercise information on the basis of the measurement data of the activity amount, the arithmetic processing load required for generating the first guidance report can be reduced in comparison with the case where the measurement data of the activity amount itself is acquired as the first exercise information.

### Supplementary Note 4

The information processing device according to supplementary note 3, in which the exercise information acquiring unit acquires at least one of an exercise intensity and an exercise frequency as data representing the exercise pattern based on the measurement data of the activity amount.

According to the information processing device according to supplementary note 4, by acquiring at least one of the exercise intensity and the exercise frequency as data representing the exercise pattern on the basis of the measurement data of the activity amount, more precise exercise patterns can be acquired.

### Supplementary Note 5

The information processing device according to supplementary note 4, in which the measurement data of the activity amount includes at least measurement data of a heart rate, and the exercise information acquiring unit determines at least one of the exercise intensity and the exercise frequency based on the measurement data of the heart rate and predetermined criteria.

According to the information processing device according to supplementary note 5, by determining at least one of the exercise intensity and the exercise frequency on the basis of the heart rate measurement data and predetermined criteria, at least one of the exercise intensity and the exercise frequency can be acquired through simple arithmetic processing.

### Supplementary Note 6

The information processing device according to supplementary note 1 or 2, in which the exercise information acquiring unit acquires measurement data of an activity amount obtained from a measurement device worn on the subject person as the first exercise information.

According to the information processing device according to supplementary note 6, the measurement data of the activity amount itself obtained by the measurement device worn on the subject person is acquired as the first exercise information, and thus the measurement data of the activity amount can be used as it is in the information processing device.

### Supplementary Note 7

The information processing device according to supplementary note 1, in which the first exercise information is information related to an exercise performed in a period before the subject person receives the first guidance report, the exercise information acquiring unit acquires second exercise information related to an exercise performed in a period between when the subject person receives the first guidance report and when the subject person takes a second health checkup, the diagnosis information acquiring unit acquires second diagnosis information including a result of the second health checkup of the subject person, the goal setting unit sets a second weight loss goal of the subject person based on the second diagnosis information, the storage unit stores in advance a second trained model, the second trained model being configured to output, as a content of second guidance for the subject person, a second advice candidate expected to be valid to achieve the second weight loss goal among a plurality of second advice candidates based on a combination of the first exercise information and the second exercise information when the first exercise information, the second exercise information, and the second weight loss goal are input, and the report generation unit inputs the first exercise information, the second exercise information, and the second weight loss goal to the second trained model, and generates a second guidance report including the content of the second guidance output from the second trained model.

The information processing device according to supplementary note 7 inputs the first exercise information, the second exercise information and the second weight loss goal of the subject person to the second trained model, and generates the second guidance report including the content of the second guidance output from the second trained model. The second trained model is a mathematical model generated through machine learning in which when the first exercise information, the second exercise information and the second weight loss goal of the subject person are input, the second advice candidate expected to be valid to achieve the second weight loss goal among the plurality of second advice candidates based on the combination of the first exercise information and the second exercise information is output as the content of the second guidance for the subject person. Thus, regardless of the first exercise information, the second exercise information and the second weight loss goal input to the second trained model, the correct content of the second guidance, i.e., the content of the second guidance that has succeeded in achieving the second weight loss goal in the past history is always output from second trained model.

Therefore, according to the information processing device according to supplementary note 7, the second guidance report including the content of the second guidance that is always effective for the first exercise information, the second exercise information and the second weight loss goal of the subject person can be provided to the subject person regardless of the view of the counselor.

### Supplementary Note 8

The information processing device according to supplementary note 1 further includes a symptom determination unit configured to determine whether an obesity symptom of the subject person is severe or mild based on the first diagnosis information, in which the storage unit stores in advance a third trained model, the third trained model being configured to output, as a content of third guidance for the subject person, a third advice candidate expected to be valid to achieve the first weight loss goal among a plurality of third advice candidates based on a combination of the first exercise information and the determination result of the obesity symptom when the first exercise information, the determination result of the obesity symptom, and the first weight loss goal are input, and the report generation unit inputs the first exercise information, the determination result of the obesity symptom, and the first weight loss goal to the third trained model, and generates a third guidance report including the content of the third guidance output from the third trained model.

The information processing device according to supplementary note 8 inputs the first exercise information, the determination result of the obesity symptom and the first weight loss goal of the subject person to the third trained model, and generates the third guidance report including the content of the third guidance output from the third trained model. The third trained model is a mathematical model generated through machine learning in which when the first exercise information, the determination result of the obesity symptom and the first weight loss goal of the subject person are input, the third advice candidate expected to be valid to achieve the first weight loss goal among the plurality of the third advice candidates based on the combination of the first exercise information and the determination result of the obesity symptom is output as the content of the third guidance for the subject person. Thus, regardless of the first exercise information, the determination result of the obesity symptom and the first weight loss goal input to the third trained model, the correct content of the third guidance, i.e., the content of the third guidance that has succeeded in achieving the first weight loss goal in the past history is always output from third trained model.

Therefore, according to the information processing device according to supplementary note 8, the third guidance report including the content of the third guidance that is always effective for the first exercise information, the determination result of the obesity symptom and the first weight loss goal of the subject person can be provided to the subject person regardless of the view of the counselor.

### Supplementary Note 9

The information processing device according to supplementary note 8, in which the first diagnosis information includes, as a result of the first health checkup, a body mass index (BMI) of the subject person, and the symptom determination unit determines that the obesity symptom is severe when the BMI is a predetermined value or greater, and determines that the obesity symptom is mild when the BMI is smaller than the predetermined value.

According to the information processing device according to supplementary note 9, by determining that the obesity symptom is severe when the BMI of the subject person is the predetermined value or greater, and determining that the obesity symptom is mild when the BMI is smaller than the predetermined value, the obesity symptom of the subject person can be determined through a simple process.

### Supplementary Note 10

A program configured to cause a computer to execute acquiring first exercise information related to an exercise performed by a subject person who receives a health guidance, acquiring first diagnosis information including a result of a first health checkup of the subject person, setting a first weight loss goal of the subject person based on the first diagnosis information, reading from a storage unit a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input, and inputting the first exercise information and the first weight loss goal to the first trained model, and generating a first guidance report including the content of the first guidance output from the first trained model.

According to the program according to supplementary note 10, the first guidance report including the content of the first guidance that is always effective for the first exercise information and the first weight loss goal of the subject person can be provided to the subject person regardless of the view of the counselor.

What is claimed is:

1. An information processing device comprising:
an exercise information acquiring unit configured to acquire first exercise information related to an exercise performed by a subject person who receives health guidance;
a diagnosis information acquiring unit configured to acquire first diagnosis information including a result of a first health checkup of the subject person;
a goal setting unit configured to set a first weight loss goal of the subject person based on the first diagnosis information;
a storage unit configured to store in advance a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input; and
a report generation unit configured to input the first exercise information and the first weight loss goal to the first trained model, and generate a first guidance report including the content of the first guidance output from the first trained model.

2. The information processing device according to claim 1, wherein
the first diagnosis information includes, as a result of the first health checkup, diagnosis results of a waist circumference, a neutral fat, a fasting blood glucose level, and a blood pressure of the subject person, and
the goal setting unit sets the first weight loss goal such that the diagnosis results of the waist circumference, the neutral fat, the fasting blood glucose level, and the blood pressure are smaller than diagnosis reference values for metabolic syndrome.

3. The information processing device according to claim 1, wherein the exercise information acquiring unit acquires an exercise pattern of the subject person as the first exercise information based on measurement data of an activity amount obtained from a measurement device worn on the subject person.

4. The information processing device according to claim 3, wherein the exercise information acquiring unit acquires at least one of an exercise intensity and an exercise frequency as data representing the exercise pattern based on the measurement data of the activity amount.

5. The information processing device according to claim 4, wherein
the measurement data of the activity amount includes at least measurement data of a heart rate, and
the exercise information acquiring unit determines at least one of the exercise intensity and the exercise frequency based on the measurement data of the heart rate and predetermined criteria.

6. The information processing device according to claim 1, wherein the exercise information acquiring unit acquires measurement data of an activity amount obtained from a measurement device worn on the subject person as the first exercise information.

7. The information processing device according to claim 1, wherein
the first exercise information is information related to an exercise performed in a period before the subject person receives the first guidance report,
the exercise information acquiring unit acquires second exercise information related to an exercise performed in a period between when the subject person receives the first guidance report and when the subject person takes a second health checkup,
the diagnosis information acquiring unit acquires second diagnosis information including a result of the second health checkup of the subject person,
the goal setting unit sets a second weight loss goal of the subject person based on the second diagnosis information,
the storage unit stores in advance a second trained model, the second trained model being configured to output, as a content of second guidance for the subject person, a second advice candidate expected to be valid to achieve the second weight loss goal among a plurality of second advice candidates based on a combination of the first exercise information and the second exercise information when the first exercise information, the second exercise information, and the second weight loss goal are input, and
the report generation unit inputs the first exercise information, the second exercise information, and the second weight loss goal to the second trained model, and generates a second guidance report including the content of the second guidance output from the second trained model.

8. The information processing device according to claim 1, further comprising a symptom determination unit configured to determine whether an obesity symptom of the subject person is severe or mild based on the first diagnosis information, wherein
the storage unit stores in advance a third trained model, the third trained model being configured to output, as a content of third guidance for the subject person, a third advice candidate expected to be valid to achieve the first weight loss goal among a plurality of third advice candidates based on a combination of the first exercise information and the determination result of the obesity symptom when the first exercise information, the determination result of the obesity symptom, and the first weight loss goal are input, and
the report generation unit inputs the first exercise information, the determination result of the obesity symptom, and the first weight loss goal to the third trained model, and generates a third guidance report including the content of the third guidance output from the third trained model.

9. The information processing device according to claim 8, wherein
the first diagnosis information includes, as a result of the first health checkup, a body mass index (BMI) of the subject person, and
the symptom determination unit determines that the obesity symptom is severe when the BMI is a predetermined value or greater, and determines that the obesity symptom is mild when the BMI is smaller than the predetermined value.

10. A non-transitory computer-readable storage medium storing a program configured to cause a computer to execute:
acquiring first exercise information related to an exercise performed by a subject person who receives a health guidance;
acquiring first diagnosis information including a result of a first health checkup of the subject person;
setting a first weight loss goal of the subject person based on the first diagnosis information;
reading from a storage unit a first trained model, the first trained model being configured to output, as a content of first guidance for the subject person, a first advice candidate expected to be valid to achieve the first weight loss goal among a plurality of first advice candidates based on the first exercise information when the first exercise information and the first weight loss goal are input; and
inputting the first exercise information and the first weight loss goal to the first trained model, and generating a first guidance report including the content of the first guidance output from the first trained model.
